SEQUENCE LISTING (107 sequences)

SEQ ID NO: 1 - DNA, 20 bp, Artificial Sequence
  IDP3952 forward primer
gtgtcgaggagatcgtcagg

SEQ ID NO: 2 - DNA, 20 bp, Artificial Sequence
  IDP3952 reverse primer
ctcccagctcaaggtagtgc

SEQ ID NO: 3 - DNA, 19 bp, Artificial Sequence
  IDP8680 forward primer
ggtcccttgatgtccatgc

SEQ ID NO: 4 - DNA, 20 bp, Artificial Sequence
  IDP8680 reverse primer
gaagcaggtcgtgatgttcc

SEQ ID NO: 5 - DNA, 21 bp, Artificial Sequence
  MZA3156 forward primer
cgaacagcaagagcttctgaa

SEQ ID NO: 6 - DNA, 22 bp, Artificial Sequence
  MZA3156 reverse primer
gtgcattggttattattctagg

SEQ ID NO: 7 - DNA, 21 bp, Artificial Sequence
  MZA5988 forward primer
cacctcattcataaaggaagg

SEQ ID NO: 8 - DNA, 19 bp, Artificial Sequence
  MZA5988 reverse primer
ctccctcntaggctccgtc

SEQ ID NO: 9 - DNA, 22 bp, Artificial Sequence
  MZA5988 forward nest primer
agaaagacatatcccaaacttg

SEQ ID NO: 10 - DNA, 19 bp, Artificial Sequence
  MZA5988 reverse nest primer
ctctctttccttatccccg

SEQ ID NO: 11 - DNA, 22 bp, Artificial Sequence
  c0176e15_21 Forward primer
cttcttcgatccttcgacggag

SEQ ID NO: 12 - DNA, 20 bp, Artificial Sequence
  c0176e15_21 Reverse primer
tcttggcgagggtggtcttc

SEQ ID NO: 13 - DNA, 20 bp, Artificial Sequence
  c0176e15_21 Forward Nest primer
aaggcgctcgagaagattca

SEQ ID NO: 14 - DNA, 20 bp, Artificial Sequence
  c0176e15_21 Revere Nest primer
gctgacgaagcgaaacatgg

SEQ ID NO: 15 - DNA, 22 bp, Artificial Sequence
  c0176e15_22 Forward primer
agagcacactggaggacctcgt

SEQ ID NO: 16 - DNA, 20 bp, Artificial Sequence
  c0176e15_22 Reverse primer
tgtgcacggccttcatctgc

SEQ ID NO: 17 - DNA, 20 bp, Artificial Sequence
  c0176e15_22 Forward Nest primer
gccgagatgaagcgcatcac

SEQ ID NO: 18 - DNA, 20 bp, Artificial Sequence
  c0176e15_22 Reverse Nest primer
cagcaggaccttctttcccg

SEQ ID NO: 19 - DNA, 20 bp, Artificial Sequence
  c0176e15_23 Forward primer
gaagaccaccctcgccaaga

SEQ ID NO: 20 - DNA, 23 bp, Artificial Sequence
  c0176e15_23 Reverse primer
cctgcccaagactacctgttcaa

SEQ ID NO: 21 - DNA, 22 bp, Artificial Sequence
  c0176e15_23 Forward Nest primer
gacgagttcgacctcagggtgt

SEQ ID NO: 22 - DNA, 20 bp, Artificial Sequence
  c0176e15_23 Reverse Nest primer
tttggaacgtacgatgccca

SEQ ID NO: 23 - DNA, 23 bp, Artificial Sequence
  c0176e15_24 Forward primer
gatgcttcttttccgtgtcttca

SEQ ID NO: 24 - DNA, 23 bp, Artificial Sequence
  c0176e15_24 Reverse primer
ggttgcgcatgactagttctctg

SEQ ID NO: 25 - DNA, 24 bp, Artificial Sequence
  c0176e15_24 Forward Nest primer
atggcaacaacattactgtggttt

SEQ ID NO: 26 - DNA, 22 bp, Artificial Sequence
  c0176e15_24 Reverse Nest primer
cactgtttcaaatgaggcggta

SEQ ID NO: 27 - DNA, 25 bp, Artificial Sequence
  c0176e15_25 Forward primer
tatgtttgaacaggtagtcttgggc

SEQ ID NO: 28 - DNA, 23 bp, Artificial Sequence
  c0176e15_25 Reverse primer
tatgcagtctcactatgctccca

SEQ ID NO: 29 - DNA, 24 bp, Artificial Sequence
  c0176e15_25 Forward Nest primer
gacattggtatggagatcgtcaca

SEQ ID NO: 30 - DNA, 20 bp, Artificial Sequence
  c0176e15_25 Reverse Nest primer
atcacacagagaaggcggca

SEQ ID NO: 31 - DNA, 23 bp, Artificial Sequence
  c0176e15_26 Forward primer
attacgatcagtcaggatgcacg

SEQ ID NO: 32 - DNA, 20 bp, Artificial Sequence
  c0176e15_26 Reverse primer
tgtgtccgaagaagccgatt

SEQ ID NO: 33 - DNA, 22 bp, Artificial Sequence
  c0176e15_26 Forward Nest primer
atgcatgaccttcttcgatcgt

SEQ ID NO: 34 - DNA, 20 bp, Artificial Sequence
  c0176e15_26 Reverse Nest primer
ggtctgctttcttggccaca

SEQ ID NO: 35 - DNA, 25 bp, Artificial Sequence
  c0176e15_27 Forward primer
gactcgattggagacttgaggtatc

SEQ ID NO: 36 - DNA, 20 bp, Artificial Sequence
  c0176e15_27 Reverse primer
cacaagccctgtggggaaat

SEQ ID NO: 37 - DNA, 25 bp, Artificial Sequence
  c0176e15_27 Forward Nest primer
cagtacattggtctgctaaactgca

SEQ ID NO: 38 - DNA, 23 bp, Artificial Sequence
  c0176e15_27 Reverse Nest primer
gaacagcatccgagggtctattt

SEQ ID NO: 39 - DNA, 25 bp, Artificial Sequence
  c0176e15_28 Forward primer
taataacacacagcaggagaaacgt

SEQ ID NO: 40 - DNA, 20 bp, Artificial Sequence
  c0176e15_28 Reverse primer
tcgcaccccttgttgtttgt

SEQ ID NO: 41 - DNA, 25 bp, Artificial Sequence
  c0176e15_28 Forward Nest primer
ttgaggatgtctttgatgagctctg

SEQ ID NO: 42 - DNA, 20 bp, Artificial Sequence
  c0176e15_28 Reverse Nest primer
tggaaacagttctgcggcat

SEQ ID NO: 43 - DNA, 22 bp, Artificial Sequence
  c0176e15_29 Forward primer
acaaggaacttgagcaggcaat

SEQ ID NO: 44 - DNA, 25 bp, Artificial Sequence
  c0176e15_29 Reverse primer
ctgcttttcataaataccgtgtcaa

SEQ ID NO: 45 - DNA, 24 bp, Artificial Sequence
  c0176e15_29 Forward Nest primer
caaagtcacaaaatgcaagctcaa

SEQ ID NO: 46 - DNA, 24 bp, Artificial Sequence
  c0176e15_29 Reverse Nest primer
tggagtaggcttattgcagaaaga

SEQ ID NO: 47 - DNA, 3747 bp, Zea mays
atggctatgatcctagacgccttcgtgcccatgctggggcgaatggtcgctggtgccgtgaaggagaggctcgacacgctcctcggcgtgcccggggagatggaaaggctggagagcacactggaggacctcgtgaatgtcctcggcgacgccgagatgaagcgcatcacggacacggccgtcgacgcctgggttcgggagcttaaggacgtcatgtacgacgccgacgacgtcctcgaccggtggcagatggaggcccaggcccgcagcagcagcgacgcgcccaagcgctcgttccctggcgctggctgctgcgcgcccctcctcacgtgcttccgggacccggcgctcgctcacgccatggcggcgcagatcaaggagctgaaccggaggctggagagcgtgtgccgtcggagctccatgtttcgcttcgtcagcgcctcgtcgtccgtccccctccggcagcaactaccaccggcatccagcggcaacggtaagacgagctcggtgatcgtccacgccgatctcatcggggagaagatcgaggaggacgggaacaggctggtggaggcgctgatcgccgacgacctgcgcgagaacgtcctcgccgtgggcatcacaggcgccggcgggatcgggaagaccaccctcgccaagagggtcttcgctgaccagcgcgtgcgcgacgagttcgacctcagggtgtgggtgtgcgtgtcgcaggacgtgaacgaggctgacctgctgtggtccgtcctcgttggcgccggaggcggccaccagctccagcagcagcacgacgccacgccggacaggtcgtcgctggagcccgcgctccagcgggccgtctcgggaaagaaggtcctgctggtgctggacgacgtgtggagcgacgtggcctggaaggaggtgctacagaacgcgttcagggccggcgctcgtggtggcagcagggtgctcgtcacgacgaggaaagagacggttgccaggcagatgaaggccgtgcacatccaccgcgtggagaagctgcagcctgaagatgggtggcgcttactcaagaaccaggttagggactggaatggatgcttcttttccgtgtcttcaaattaactatggcaacaacattactgtggtttgatgatgcgtaaaagcaccagttaaattacacttgcactctaaaggcattcaaccacttgattgatgtcatgtcagccaactgaacttttatctcctctaaattcagaaggagatgtccaccatcctcaccagcacgattaaacatagggcacttggtgaacaatctcaccccccttcttttcagcttcactctcaaggctaaactataatacacaattcgccaaagaaaatgtttaactttgtttgggcatcgtacgttccaaatcccacgccgaaattcttataaacaaacacacctaattttacatgacaaaagaatactcttttgagagagagaaaactggacactggctactactgatgcatccagagaggacttgaaccatctagtacatggtctgatggcagacatacaagattaatttgagcacagttgttatacaaattcttaaccattcttttatgtttgaacaggtagtcttgggcaggaatccaaccgacattgaaaatttcaaagacattggtatggagatcgtcacaagatgcgattgcctgccgcttgccatcaagacagtgggtgggctactgtgcacaaaagagagaacgttcagagactgggaggaagtctcaaggagcgccgcatggtccgtggcaggactacctgaagaggttcacaacgccatctacctgagctatgccgatctaccgcctcatttgaaacagtgtttcctgcactgctcccttttcccaaaagacgaagtcatcaaacgggtggatgtcgttcagatgtggatcgccgaggggtttgtacaagaggatgggtcctcagcgctgctcgaagatgtagggaacatgtattacagagaactagtcatgcgcaacctactcgaacctgatggccagtattacgatcagtcaggatgcacgatgcatgaccttcttcgatcgtttgccaattatctggcaaaagatgaagcgttgcttcttacgcagggccaaagcttatgcgacatgaagacaaaagccaagctgcgtcggctgtccgtagcaaccgaaaatgtgctccaaagtaccttcagaaatcagaagcagctgagggcgctaatgatactccgaagcaccacggttcagctggaagagttcctgcatgacctgcctaagctgcgactgctgcatctcgggggtgtaaacctcacaaccttgccgccttctctgtgtgatctgaagcatctaagatacttggagctgtctggtaccatgatagatgcaatcccagactcgattggagacttgaggtatctacagtacattggtctgctaaactgcataaatctgttcagtcttcctgggagcatagtgagactgcataggctgagagctctccacatcaagggggccagtgtgaatgacatccccagggggatagggagattacaaaaccttgtcgagctgactggttttttaacacagaatgatgctgctgcaggttggaacagcctggaggagctaggccacctgccccagctcagcctcttgtatctaagcaacctagagaaagcacacaccggctctgtggccaagaaagcaggcctccaaggcaagcgccaccttagatacttaagcttggagtgcacaccaagagccgctggtggaaatcagatcaaagataataacacacagcaggagaaacgtcagattgaggatgtctttgatgagctctgtccaccggtttgcctcgaaaacctctcactaatcggcttcttcggacacaagcttcctaaatggatgagctcaggcgagatggatcttaagtacctaagatcaataaaactcgaagattgcacctactgcgagcagctccccgcattgggccatctcctgagtttagatttcctgctgatcaaacatgcgccatctattatgagaattggacacgaattcttttgcagcagcaatgctacacaaatagaccctcggatgctgttcccaaggctggagaaacttggatttgataggttggatggatgggaagaatggatatgggacaaggaacttgagcaggcaatgccaaacatcttttctctcaaagtcacaaaatgcaagctcaagtatttccccacagggcttgtgcatcaaaccaggaccttgagagaattgatcatatctgaagcttgcaacttgacatcagttgcaaactttctcctcctcagcgatctgcatctccatgccaacccaaatctcgagatgatcgccaatctccctaaactacgaaggctttcggttatccaatgccccaagttgaatgcacttgtgggtttaacagaactgcaaagcattacattgcaggactatgccgcagaactgtttccacagtacttggaagaaactagtgctgcaaagctagaggttttctgtaatgaagaactcttcaaacttataaccctgcaagaaggttcagagtggtgcaagatcaagaatatccaaaatgttaaagcatatgctcccaaaggaggcgaccgtaaaggatggtacgcattatacactaaggaaccgtttagcctgaccacaaacaacaaggggtgcgaaatatttgaagttgcaaagtcctaa

SEQ ID NO: 48 - DNA, 3201 bp, Zea mays
atggctatgatcctagacgccttcgtgcccatgctggggcgaatggtcgctggtgccgtgaaggagaggctcgacacgctcctcggcgtgcccggggagatggaaaggctggagagcacactggaggacctcgtgaatgtcctcggcgacgccgagatgaagcgcatcacggacacggccgtcgacgcctgggttcgggagcttaaggacgtcatgtacgacgccgacgacgtcctcgaccggtggcagatggaggcccaggcccgcagcagcagcgacgcgcccaagcgctcgttccctggcgctggctgctgcgcgcccctcctcacgtgcttccgggacccggcgctcgctcacgccatggcggcgcagatcaaggagctgaaccggaggctggagagcgtgtgccgtcggagctccatgtttcgcttcgtcagcgcctcgtcgtccgtccccctccggcagcaactaccaccggcatccagcggcaacggtaagacgagctcggtgatcgtccacgccgatctcatcggggagaagatcgaggaggacgggaacaggctggtggaggcgctgatcgccgacgacctgcgcgagaacgtcctcgccgtgggcatcacaggcgccggcgggatcgggaagaccaccctcgccaagagggtcttcgctgaccagcgcgtgcgcgacgagttcgacctcagggtgtgggtgtgcgtgtcgcaggacgtgaacgaggctgacctgctgtggtccgtcctcgttggcgccggaggcggccaccagctccagcagcagcacgacgccacgccggacaggtcgtcgctggagcccgcgctccagcgggccgtctcgggaaagaaggtcctgctggtgctggacgacgtgtggagcgacgtggcctggaaggaggtgctacagaacgcgttcagggccggcgctcgtggtggcagcagggtgctcgtcacgacgaggaaagagacggttgccaggcagatgaaggccgtgcacatccaccgcgtggagaagctgcagcctgaagatgggtggcgcttactcaagaaccaggtagtcttgggcaggaatccaaccgacattgaaaatttcaaagacattggtatggagatcgtcacaagatgcgattgcctgccgcttgccatcaagacagtgggtgggctactgtgcacaaaagagagaacgttcagagactgggaggaagtctcaaggagcgccgcatggtccgtggcaggactacctgaagaggttcacaacgccatctacctgagctatgccgatctaccgcctcatttgaaacagtgtttcctgcactgctcccttttcccaaaagacgaagtcatcaaacgggtggatgtcgttcagatgtggatcgccgaggggtttgtacaagaggatgggtcctcagcgctgctcgaagatgtagggaacatgtattacagagaactagtcatgcgcaacctactcgaacctgatggccagtattacgatcagtcaggatgcacgatgcatgaccttcttcgatcgtttgccaattatctggcaaaagatgaagcgttgcttcttacgcagggccaaagcttatgcgacatgaagacaaaagccaagctgcgtcggctgtccgtagcaaccgaaaatgtgctccaaagtaccttcagaaatcagaagcagctgagggcgctaatgatactccgaagcaccacggttcagctggaagagttcctgcatgacctgcctaagctgcgactgctgcatctcgggggtgtaaacctcacaaccttgccgccttctctgtgtgatctgaagcatctaagatacttggagctgtctggtaccatgatagatgcaatcccagactcgattggagacttgaggtatctacagtacattggtctgctaaactgcataaatctgttcagtcttcctgggagcatagtgagactgcataggctgagagctctccacatcaagggggccagtgtgaatgacatccccagggggatagggagattacaaaaccttgtcgagctgactggttttttaacacagaatgatgctgctgcaggttggaacagcctggaggagctaggccacctgccccagctcagcctcttgtatctaagcaacctagagaaagcacacaccggctctgtggccaagaaagcaggcctccaaggcaagcgccaccttagatacttaagcttggagtgcacaccaagagccgctggtggaaatcagatcaaagataataacacacagcaggagaaacgtcagattgaggatgtctttgatgagctctgtccaccggtttgcctcgaaaacctctcactaatcggcttcttcggacacaagcttcctaaatggatgagctcaggcgagatggatcttaagtacctaagatcaataaaactcgaagattgcacctactgcgagcagctccccgcattgggccatctcctgagtttagatttcctgctgatcaaacatgcgccatctattatgagaattggacacgaattcttttgcagcagcaatgctacacaaatagaccctcggatgctgttcccaaggctggagaaacttggatttgataggttggatggatgggaagaatggatatgggacaaggaacttgagcaggcaatgccaaacatcttttctctcaaagtcacaaaatgcaagctcaagtatttccccacagggcttgtgcatcaaaccaggaccttgagagaattgatcatatctgaagcttgcaacttgacatcagttgcaaactttctcctcctcagcgatctgcatctccatgccaacccaaatctcgagatgatcgccaatctccctaaactacgaaggctttcggttatccaatgccccaagttgaatgcacttgtgggtttaacagaactgcaaagcattacattgcaggactatgccgcagaactgtttccacagtacttggaagaaactagtgctgcaaagctagaggttttctgtaatgaagaactcttcaaacttataaccctgcaagaaggttcagagtggtgcaagatcaagaatatccaaaatgttaaagcatatgctcccaaaggaggcgaccgtaaaggatggtacgcattatacactaaggaaccgtttagcctgaccacaaacaacaaggggtgcgaaatatttgaagttgcaaagtcctaa

SEQ ID NO: 49 - protein, 1066 aa, Zea mays
MAMILDAFVPMLGRMVAGAVKERLDTLLGVPGEMERLESTLEDLVNVLGDAEMKRITDTAVDAWVRELKDVMYDADDVLDRWQMEAQARSSSDAPKRSFPGAGCCAPLLTCFRDPALAHAMAAQIKELNRRLESVCRRSSMFRFVSASSSVPLRQQLPPASSGNGKTSSVIVHADLIGEKIEEDGNRLVEALIADDLRENVLAVGITGAGGIGKTTLAKRVFADQRVRDEFDLRVWVCVSQDVNEADLLWSVLVGAGGGHQLQQQHDATPDRSSLEPALQRAVSGKKVLLVLDDVWSDVAWKEVLQNAFRAGARGGSRVLVTTRKETVARQMKAVHIHRVEKLQPEDGWRLLKNQVVLGRNPTDIENFKDIGMEIVTRCDCLPLAIKTVGGLLCTKERTFRDWEEVSRSAAWSVAGLPEEVHNAIYLSYADLPPHLKQCFLHCSLFPKDEVIKRVDVVQMWIAEGFVQEDGSSALLEDVGNMYYRELVMRNLLEPDGQYYDQSGCTMHDLLRSFANYLAKDEALLLTQGQSLCDMKTKAKLRRLSVATENVLQSTFRNQKQLRALMILRSTTVQLEEFLHDLPKLRLLHLGGVNLTTLPPSLCDLKHLRYLELSGTMIDAIPDSIGDLRYLQYIGLLNCINLFSLPGSIVRLHRLRALHIKGASVNDIPRGIGRLQNLVELTGFLTQNDAAAGWNSLEELGHLPQLSLLYLSNLEKAHTGSVAKKAGLQGKRHLRYLSLECTPRAAGGNQIKDNNTQQEKRQIEDVFDELCPPVCLENLSLIGFFGHKLPKWMSSGEMDLKYLRSIKLEDCTYCEQLPALGHLLSLDFLLIKHAPSIMRIGHEFFCSSNATQIDPRMLFPRLEKLGFDRLDGWEEWIWDKELEQAMPNIFSLKVTKCKLKYFPTGLVHQTRTLRELIISEACNLTSVANFLLLSDLHLHANPNLEMIANLPKLRRLSVIQCPKLNALVGLTELQSITLQDYAAELFPQYLEETSAAKLEVFCNEELFKLITLQEGSEWCKIKNIQNVKAYAPKGGDRKGWYALYTKEPFSLTTNNKGCEIFEVAKS

SEQ ID NO: 50 - DNA, 3794 bp, Zea mays
atggctatgatcctagacgccttcgtgcccatgctggggcgaatggtcgctggtgccgtgaaggagaggctcgacacgctcctcggcgtgcccggggagatggaaaggctggagagcacactggaggacctcgtgaatgtcctcggcgacgccgagatgaagcgcatcacggacacggccgtcgacgcctgggttcgggagcttaaggacgtcatgtacgacgccgacgacgtcctcgaccggtggcagatggaggcccaggcccgcagcagcagcgacgcgcccaagcgctcgttccctggcgctggctgctgcgcgcccctcctcacgtgcttccgggacccggcgctcgctcacgccatggcggcgcagatcaaggagctgaaccggaggctggagagcgtgtgccgtcggagctccatgtttcgcttcgtcagcgcctcgtcgtccgtccccctccggcagcaactaccaccggcatccagcggcaacggtaagacgagctcggtgatcgtccacgccgatctcatcggggagaagatcgaggaggacgggaacaggctggtggaggtgctgatcgctgacgacctgcgcgagaacgtcctcgccgtgggcatcacaggcgccggcgggatcgggaagaccaccctcgccaagagggtcttcgctgaccagcgcgtgcgcgacgagttcgacctcagggtgtgggtgtgcgtgtcgcaggacgtgaacgaggctgacctgctgtggtccgtcctcgttggcgccggaggcggccaccagctccagcagcagcacgacgccacgccggacaggtcgtcgctctggagcccgcgctccagcgggccgtctcgggaaagaaggtcctgctggtgctggacgacgtgtggagcgacgtggcctggaaggaggtgctccagaacgcgttcagggccggcgctcgtggcggcagcagggtgctcgtcacgacgaggaaagagtcggttgccaggcagatgaaggccgtgcacatccaccgcgtggagaagctgcagcctgaagatgggtggcgcttactcaagaaccaggttagggactggaatgaatgcttcttttccgtgtcttcaaattaactatggcgacaacattactgcggtttgatgatgcgtaaaagcaccagttaaattacacttgcactctaaagtcattcaaccatttaattgatgtcagccaactgaacttttatctcctctaaattcagtgttctgacttgcatttttttttttaaaaaaaaaggagatgtccattgtccaccatcctcaccagcacaattaaacaaattaaacatagggcacttggtgaacaatctcacccccttcttttcagcttcactctcaaggctaaactataatacacaattcgccaaagaaaatgtttaactttgtttggacatcgtacgttccaaatcccacgccaaaattcttataaacaaacacagctaattttacatgacaaaagaaggctcttttgagagagagaaaactggacactggctactgactgatgcatccagagaggacttgaaccatctagtacatggtctgatggcagacatacaagattaatttgagcacagttgttatacaaatacttaaccatactttatgtttgaacaggtagtcttgggcaggaatccaaccgacatagaaaatttcaaagacattggtatggagatcgtcacaagatgcgattgcctgccgcttgccatcaagacagtgggtgggctactgtgcacaaaagagagaacgttcagagactgggaggaagtctcaaggagcgccgcatggtccgtggcaggactacctgaagaggttcacaacgccatctacctgagctatgccgatctaccgcctcatttgaaacagtgtttcctgcactgctcccttttcccaaaagacgaagtcatcaaacgggtggatgtcgttcagatgtggatcgccgaggggtttgtacaagaggatgggtcctcagcgctgctcgaagatgtagggaacatgtattacagagaactagtcatgcgcaacctactcgaacctgatggccagtattacgatcagtcaggatgcacgatgcatgaccttcttcgatcgtttgccaattatctggcaaaagatgaagcgttgcttcttacgcagggccaaagtttatgcgacatgaagacaaaagccaagctgcgtcggctgtccgtagccaccgaaaatgtgctccaaagtaccttcagaaatcagaagcagctgagggcgctaatgatactccgaagcaccacggttcagctggaagagttcctgcatgacctgcctaagctgcgactgctgcatctcgggggtgtaaacctcacaaccttgccgccttctctgtgtgatctgaagcatctaagatacttggagctgtctggtaccatgatagatgcaatcccagactcgattggagacttgaggtatctacagtacattggtctgctaaactgcataaatctgttcagtcttcctgggagcatagtgaggctgcataagctgagagctctccacatcaagggggccagtgtgaatgacgacatccccagggggatagggagattacaaaaccttgttgagctgactggttttttaacacagaatgatgctgctgcaggttggaacagcctggaggagctaggccaccttccccagctcagcctcttgtatctaagcaacctagagaaagcacacaccggctctgtggccaagaaagcagacctccaaggcaagcgccaccttagatacttaagcttggagtgcacaccaagagccgctggtggaaatcagatcaaagataataacacacagcaggagaaacgtcagattgaggatgtctttgatgagctctgtccaccggtttgcctcgaaaacctctcactaatcggcttcttcggacacaagcttcctaaatggatgagctcaggcgagatggatcttaagtacctaagatcaataaaactcgaagattgcacctactgcgagcagctccccgcattgggccatctcctgagtttagatttcctgctgatcaaacatgcgccatctattatgagaattggacacgaattcttttgcagcagcaatgctacacaaatagaccctcggatgctgttcccaaggctggagaaacttggatttgataggttggatggatgggaagaatggatatgggacaaggaactggagcaggcaatgccaaacatcttttctctcaaagtcacaaaatgcaagctcaagtatttccccccagggcttgtgcatcaaaccagggccttgaaagaattgatcatatctgaagcttgcaacttgacatcagttgcaaactttctcctcctcagcgatctgcatctccatgccaacccaaatctcgagatgatcgctaatctccctaaactacgaaggctttcggttatccaatgccccaagttgaatgcacttgtgggtttaacagaactgcaaagcatcacattgcaggactatgccgcagaactgtttccacagtacttggaagaaactagtgctgcaaagctagaggttttctgtaatgaagaactcttcaaacttataaccctgcaagaaggttcagagtggtgcaagatcaagaatatccaaaatgttaaagcatatgctcccaaaggaggcgaccgtaaaggatggtatgcattatacactaaggaaccgtttagcctgaccacaaacaacaaggggtgcgaaatatttgaagttgcaaagtcctaa

SEQ ID NO: 51 - DNA, 3205 bp, Zea mays
atggctatgatcctagacgccttcgtgcccatgctggggcgaatggtcgctggtgccgtgaaggagaggctcgacacgctcctcggcgtgcccggggagatggaaaggctggagagcacactggaggacctcgtgaatgtcctcggcgacgccgagatgaagcgcatcacggacacggccgtcgacgcctgggttcgggagcttaaggacgtcatgtacgacgccgacgacgtcctcgaccggtggcagatggaggcccaggcccgcagcagcagcgacgcgcccaagcgctcgttccctggcgctggctgctgcgcgcccctcctcacgtgcttccgggacccggcgctcgctcacgccatggcggcgcagatcaaggagctgaaccggaggctggagagcgtgtgccgtcggagctccatgtttcgcttcgtcagcgcctcgtcgtccgtccccctccggcagcaactaccaccggcatccagcggcaacggtaagacgagctcggtgatcgtccacgccgatctcatcggggagaagatcgaggaggacgggaacaggctggtggaggtgctgatcgctgacgacctgcgcgagaacgtcctcgccgtgggcatcacaggcgccggcgggatcgggaagaccaccctcgccaagagggtcttcgctgaccagcgcgtgcgcgacgagttcgacctcagggtgtgggtgtgcgtgtcgcaggacgtgaacgaggctgacctgctgtggtccgtcctcgttggcgccggaggcggccaccagctccagcagcagcacgacgccacgccggacaggtcgtcgctctggagcccgcgctccagcgggccgtctcgggaaagaaggtcctgctggtgctggacgacgtgtggagcgacgtggcctggaaggaggtgctccagaacgcgttcagggccggcgctcgtggcggcagcagggtgctcgtcacgacgaggaaagagtcggttgccaggcagatgaaggccgtgcacatccaccgcgtggagaagctgcagcctgaagatgggtggcgcttactcaagaaccagtagtcttgggcaggaatccaaccgacatagaaaatttcaaagacattggtatggagatcgtcacaagatgcgattgcctgccgcttgccatcaagacagtgggtgggctactgtgcacaaaagagagaacgttcagagactgggaggaagtctcaaggagcgccgcatggtccgtggcaggactacctgaagaggttcacaacgccatctacctgagctatgccgatctaccgcctcatttgaaacagtgtttcctgcactgctcccttttcccaaaagacgaagtcatcaaacgggtggatgtcgttcagatgtggatcgccgaggggtttgtacaagaggatgggtcctcagcgctgctcgaagatgtagggaacatgtattacagagaactagtcatgcgcaacctactcgaacctgatggccagtattacgatcagtcaggatgcacgatgcatgaccttcttcgatcgtttgccaattatctggcaaaagatgaagcgttgcttcttacgcagggccaaagtttatgcgacatgaagacaaaagccaagctgcgtcggctgtccgtagccaccgaaaatgtgctccaaagtaccttcagaaatcagaagcagctgagggcgctaatgatactccgaagcaccacggttcagctggaagagttcctgcatgacctgcctaagctgcgactgctgcatctcgggggtgtaaacctcacaaccttgccgccttctctgtgtgatctgaagcatctaagatacttggagctgtctggtaccatgatagatgcaatcccagactcgattggagacttgaggtatctacagtacattggtctgctaaactgcataaatctgttcagtcttcctgggagcatagtgaggctgcataagctgagagctctccacatcaagggggccagtgtgaatgacgacatccccagggggatagggagattacaaaaccttgttgagctgactggttttttaacacagaatgatgctgctgcaggttggaacagcctggaggagctaggccaccttccccagctcagcctcttgtatctaagcaacctagagaaagcacacaccggctctgtggccaagaaagcagacctccaaggcaagcgccaccttagatacttaagcttggagtgcacaccaagagccgctggtggaaatcagatcaaagataataacacacagcaggagaaacgtcagattgaggatgtctttgatgagctctgtccaccggtttgcctcgaaaacctctcactaatcggcttcttcggacacaagcttcctaaatggatgagctcaggcgagatggatcttaagtacctaagatcaataaaactcgaagattgcacctactgcgagcagctccccgcattgggccatctcctgagtttagatttcctgctgatcaaacatgcgccatctattatgagaattggacacgaattcttttgcagcagcaatgctacacaaatagaccctcggatgctgttcccaaggctggagaaacttggatttgataggttggatggatgggaagaatggatatgggacaaggaactggagcaggcaatgccaaacatcttttctctcaaagtcacaaaatgcaagctcaagtatttccccccagggcttgtgcatcaaaccagggccttgaaagaattgatcatatctgaagcttgcaacttgacatcagttgcaaactttctcctcctcagcgatctgcatctccatgccaacccaaatctcgagatgatcgctaatctccctaaactacgaaggctttcggttatccaatgccccaagttgaatgcacttgtgggtttaacagaactgcaaagcatcacattgcaggactatgccgcagaactgtttccacagtacttggaagaaactagtgctgcaaagctagaggttttctgtaatgaagaactcttcaaacttataaccctgcaagaaggttcagagtggtgcaagatcaagaatatccaaaatgttaaagcatatgctcccaaaggaggcgaccgtaaaggatggtatgcattatacactaaggaaccgtttagcctgaccacaaacaacaaggggtgcgaaatatttgaagttgcaaagtcctaa

SEQ ID NO: 52 - protein, 332 aa, Zea mays
MAMILDAFVPMLGRMVAGAVKERLDTLLGVPGEMERLESTLEDLVNVLGDAEMKRITDTAVDAWVRELKDVMYDADDVLDRWQMEAQARSSSDAPKRSFPGAGCCAPLLTCFRDPALAHAMAAQIKELNRRLESVCRRSSMFRFVSASSSVPLRQQLPPASSGNGKTSSVIVHADLIGEKIEEDGNRLVEVLIADDLRENVLAVGITGAGGIGKTTLAKRVFADQRVRDEFDLRVWVCVSQDVNEADLLWSVLVGAGGGHQLQQQHDATPDRSSLWSPRSSGPSRERRSCWCWTTCGATWPGRRCSRTRSGPALVAAAGCSSRRGKSRLPGR

SEQ ID NO: 53 - DNA, 25 bp, Artificial Sequence
  c0176e15_45 Forward primer
ctgcatgtggactttattcagaaga

SEQ ID NO: 54 - DNA, 20 bp, Artificial Sequence
  c0176e15_46 Reverse primer
tccttttgcattggatggct

SEQ ID NO: 55 - DNA, 25 bp, Artificial Sequence
  c0176e15_45 Forward Nest primer
catcaagttttaggtggactgaagc

SEQ ID NO: 56 - DNA, 24 bp, Artificial Sequence
  c0176e15_46 Reverse Nest primer
aaatggcctgattcttttggtaga

SEQ ID NO: 57 - DNA, 24 bp, Artificial Sequence
  c0176e15_8 Forward primer
gatgcagtgtattctcgacgaact

SEQ ID NO: 58 - DNA, 20 bp, Artificial Sequence
  c0176e15_7 Reverse Nest primer
tcgatgtaatccggtgcgga

SEQ ID NO: 59 - DNA, 22 bp, Artificial Sequence
  MZA6815_Forward primer
aaatgtgaaccttgaggaacac

SEQ ID NO: 60 - DNA, 22 bp, Artificial Sequence
  MZA6815_Reverse primer
ctgaaaaccaagatagttcatg

SEQ ID NO: 61 - DNA, 18 bp, Artificial Sequence
  IDP200_Forward primer
gcatgcccgactgtatcc

SEQ ID NO: 62 - DNA, 20 bp, Artificial Sequence
  IDP200 reverse primer
cctcgtcagccagagtaacc

SEQ ID NO: 63 - DNA, 20 bp, Artificial Sequence
  MZA760 Forward primer
atgctgttctgtcactcggt

SEQ ID NO: 64 - DNA, 22 bp, Artificial Sequence
  MZA760 Reverse primer
gatagacacccttcaagcaaat

SEQ ID NO: 65 - DNA, 20 bp, Artificial Sequence
  MZA15537 Forward primer
gagcagtacgagttcatcga

SEQ ID NO: 66 - DNA, 22 bp, Artificial Sequence
  MZA15537 Reverse primer
ctcgaccacctaattaaacagt

SEQ ID NO: 67 - DNA, 3201 bp, Zea mays
atggcggtgagtaaatcgtttctgattttcgcatatttatcatgtctttgacctcttgatgtctgtagtaaaaagtccaaattactccatccaagtttgcctatcccttaaactaacatttagttcaatttactacctctaactatttcagttggtctaatttacaccttaaaaagatttttcgttttgtttctctgtgtataaattgtgtgattatagaggtcatcatagtttgtgttagaaaaatatatcatgaacgtttcatcactatattttgtatgatattatatctctagtgtcatttactattaaatttcctaagctatcaaaaatagtggcagaaaagaatatatattgttttcctaatataactcataatgtcctctatcacatcacaaaatttgatattagtacttaacttatacgtgatgaaacagaaaataacaaatcttgttaaggggtaaactggactaactcaaatagttgggtgagtaaatcgaaccaaatatcatcttagggttaattagaccaatgccaaaattgaggggagtaatttggacttttttaataaaactagagcgaaaacacgtccacttgtaaaatgtaaaaaaattctccagcctgaacaagtcaaggcagaatcgaagtatcatttacactagttcttatttaaccagacaattagtcatcacaggacagaccaattccagatttcttacagttgactcaaaatgtaatacaatatgtatgtatccgtatggaagacagagggagacataatgggacctataagtactgctgaatgagtagtgtacttagctggtttggtagttataaacccacctttgtctgttcatgtggtgattggtgtgctgtgtatgctaggcctattcatgctgtgagtgtggcagataatttcccagaataaatccttgttgaaactagtaaactgcaaatgccacaattctagacctgtgtgcacttgatccagtatatgcattatactcttcttttgcatgttagcatgtatcatagacatcccaaatatttagggggttcagtcttagttttctcagaaatgataatctcctgaatcctgctgcagggtattcagcatactttgggatgtttgagaatctttttgtctttgctaattcttctcccacaaaatgataactatcctagcaacagatggttgcttttacatttttatatatcataactcatagccctccagaaagttgactaaagctaccgattactctgctctgtacagcaacctgaaattcctgcatttggggattgggaaaccactggaaacacaccttacacacaaaagttcgaggatgcacggaagaacaagaaaacaggaattcttgcacaaccaaatgatccaaggagggatctggaacctcctcgcaagtcgcctttgcacccaacggtctacaaaactaatcctcaagaccaaggtccaaggaacccaccacatcgaccaagacctgaacttgacaaccaacgacactctgaccggcctactcaccgtgagtctgcaccacgaagacattcaaatcttcagagagaacaagggagtaatgctgggacaccaagaagcccctacagaacagctgctgggtctgcttcaccaatgcagccaaacaaccaatcaaagccaaagcacaggtcaactggaatgcagactccagagaggagggcatcttcagaggggcttggccagcatacacctggaaggagcaggatgaagccgggtggctatgaggtaataataccttactatacttcattttttcaataatatttcaatgaatgtgggctagaaatccctttagagtaattactcttgcttctgcattggtaaatccagcctgaagaagtggctgtaccaccctttggcgaatgggatgatgcaaatgcagcatcaggtgaaaagtacactggtatcttcaacagggtgagggatgataggttgtcacctacctcttctgctaggcaaccgtctactactcgtagtgaagagaataaggtgcaacaggtatgcaaaagcttgtccagagtaaagttgaaaattcagtttatatgttcaattcttcattgtatattattgcacaccacatatgcaaatgcaattatgcaaaaaagtatcataatctctatgatgttgatgctaacttaatattaagtattgtatctgatagatcatatataacaaaaatatatttatgctatttgcttttagaactctttggcatttaatcttccctatgttctccaatagaacatatcctgactatattattgcactagcatcagaaatgtccaaggttcatcttctatttttattttctctaacacgcaagtgagttacatatcattttattatcaagaaagaaaagagtacgcaagagggacaaagtccccgaaacacacccaaaactcacagccacactgcaactggaaacaaattgcccttttcaacagcaagtgatcaacaagctaccagtaaaaattaattgtctggtaatcaactgtgacatccagctatccgaccttgaatcttggtctgattactaaacaataaatgttttctatatagttcattctatatgttccttaaatatttaattttgtgaccttaagtttatttaaagtatcaatagcaatatttgtatgccatacagctggtggattggtagcgtagagaacaggttatttttatggaaaccacacacaattatatgtattctgcaagtaaaaaatattggcaagtggcatcttccttttaaaaaaaaactgccaacttcattgtcaaattcttacatgatagatatgtattaaagatatgttacataaatacatcagctgcatgatttgctaattaagtaattatcttcatttgtttgttttgattcagctagcactcaaatactgagacatattccttgttgctctatcaacagtagtaaactagtaatcgtggcattgttttggtatagaatgttgaactgttgttaagatgagttattttgccttgaatttatccagataaataaacatggaaattttaaaaacacaaatatgtttcttatttaaagttccctaaaactctgcatgtcatacagggatatcttatcgactgatccttgtctcttttatctacagaaatgttcttgctgcatactttga

SEQ ID NO: 68 - DNA, 699 bp, Zea mays
atggcgcaacctgaaattcctgcatttggggattgggaaaccactggaaacacaccttacacacaaaagttcgaggatgcacggaagaacaagaaaacaggaattcttgcacaaccaaatgatccaaggagggatctggaacctcctcgcaagtcgcctttgcacccaacggtctacaaaactaatcctcaagaccaaggtccaaggaacccaccacatcgaccaagacctgaacttgacaaccaacgacactctgaccggcctactcaccgtgagtctgcaccacgaagacattcaaatcttcagagagaacaagggagtaatgctgggacaccaagaagcccctacagaacagctgctgggtctgcttcaccaatgcagccaaacaaccaatcaaagccaaagcacaggtcaactggaatgcagactccagagaggagggcatcttcagaggggcttggccagcatacacctggaaggagcaggatgaagccgggtggctatgagcctgaagaagtggctgtaccaccctttggcgaatgggatgatgcaaatgcagcatcaggtgaaaagtacactggtatcttcaacagggtgagggatgataggttgtcacctacctcttctgctaggcaaccgtctactactcgtagtgaagagaataaggtgcaacagaaatgttcttgctgcatactttga

SEQ ID NO: 69 - protein, 232 aa, Zea mays
MAQPEIPAFGDWETTGNTPYTQKFEDARKNKKTGILAQPNDPRRDLEPPRKSPLHPTVYKTNPQDQGPRNPPHRPRPELDNQRHSDRPTHRESAPRRHSNLQREQGSNAGTPRSPYRTAAGSASPMQPNNQSKPKHRSTGMQTPERRASSEGLGQHTPGRSRMKPGGYEPEEVAVPPFGEWDDANAASGEKYTGIFNRVRDDRLSPTSSARQPSTTRSEENKVQQKCSCCIL

SEQ ID NO: 70 - DNA, 3201 bp, Zea mays
atggcggtgagtaaatcgtttctgattttcgcatatttatcatgtctttgacctcttgatgtctgtagtaaaaagtccaaattactccatccaagtttgcctatcccttaaactaacatttagttcaatttactacctctaactatttcagttggtctaatttacaccttaaaaagatttttcgttttgtttctctgtgtataaattgtgtgattatagaggtcatcatagtttgtgttagaaaaatatatcatgaacgtttcatcactatattttgtatgatattatatctctagtgtcatttactattaaatttcctaagctatcaaaaatagtggcagaaaagaatatatattgttttcctaatataactcataatgtcctctatcacatcacaaaatttgatattagtacttaacttatacgtgatgaaacagaaaataacaaatcttgttaaggggtaaactggactaactcaaatagttgggtgagtaaatcgaaccaaatatcatcttagggttaattagaccaatgccaaaattgaggggagtaatttggacttttttaataaaactagagcgaaaacacgtccacttgtaaaatgtaaaaaaattctccagcctgaacaagtcaaggcagaatcgaagtatcatttacactagttcttatttaaccagacaattagtcatcacaggacagaccaattccagatttcttacagttgactcaaaatgtaatacaatatgtatgtatccgtatggaagacagagggagacataatgggacctataagtactgctgaatgagtagtgtacttagctggtttggtagttataaacccacctttgtctgttcatgtggtgattggtgtgctgtgtatgctaggcctattcatgctgtgagtgtggcagataatttcccagaataaatccttgttgaaactagtaaactgcaaatgccacaattctagacctgtgtgcacttgatccagtatatgcattatactcttcttttgcatgttagcatgtatcatagacatcccaaatatttagggggttcagtcttagttttctcagaaatgataatctcctgaatcctgctgcagggtattcagcatactttgggatgtttgagaatctttttgtctttgctaattcttctcccacaaaatgataactatcctagcaacagatggttgcttttacatttttatatatcataactcatagccctccagaaagttgactaaagctaccgattactctgctctgtacagcaacctgaaattcctgcatttggggattgggaaaccactggaaacacactttacacacaaaagttcgaggatgcacggaagaacaagaaaacaggaattcttgcacaaccaaatgatccaaggagggatctggaacctcctcgcaagtcgcctttgcacccaacggtctacaaaactaatcctcaagaccaaggtccaaggaacccaccacatcgaccaagacctgaacttgacaaccaacgacactctgaccggcctactcaccgtgagtctgcaccacgaagacattcaaatcttcagagagaacaagggagtaatgctgggacaccaagaagcccctacagaacagctgctgggtctgcttcaccaatgcagccaaacaaccaatcaaagccaaagcacaggtcaactggaatgcagactccagagaggagggcatcttcagaggggcttggccagcatacacctggaaggagcaggatgaagccgggtggctatgaggtaataataccttactatacttcattttttcaataatatttcaatgaatgtgggctagaaatccctttagagtaattactcttgcttctgcattggtaaatccagcctgaagaagtggctgtaccaccctttggcgaatgggatgatgcaaatgcagcatcaggtgaaaagtacactggtatcttcaacagggtgagggatgataggttgtcacctacctcttctgctaggcaaccgtctactactcgtagtgaagagaataaggtgcaacaggtatgcaaaagcttgtccagagtaaagttgaaaattcagtttatatgttcaattcttcattgtatattattgcacaccacatatgcaaatgcaattatgcaaaaaagtatcataatctctatgatgttgatgctaacttaatattaagtattgtatctgatagatcatatataacaaaaatatatttatgctatttgcttttagaactctttggcatttaatcttccctatgttctccaatagaacatatcctgactatattattgcactagcatcagaaatgtccaaggttcatcttctatttttattttctctaacacgcaagtgagttacatatcattttattatcaagaaagaaaagagtacgcaagagggacaaagtccccgaaacacacccaaaactcacagccacactgcaactggaaacaaattgcccttttcaacagcaagtgatcaacaagctaccagtaaaaattaattgtctggtaatcaactgtgacatccagctatccgaccttgaatcttggtctgattactaaacaataaatgttttctatatagttcattctatatgttccttaaatatttaattttgtgaccttaagtttatttaaagtatcaatagcaatatttgtatgccatacagctggtggattggtagcgtagagaacaggttatttttatggaaaccacacacaattatatgtattctgcaagtaaaaaatattggcaagtggcatcttccttttaaaaaaaaactgccaacttcattgtcaaattcttacatgatagatatgtattaaagatatgttacataaatacatcagctgcatgatttgctaattaagtaattatcttcatttgtttgttttgattcagctagcactcaaatactgagacatattccttgttgctctatcaacagtagtaaactagtaatcgtggcattgttttggtatagaatgttgaactgttgttaagatgagttattttgccttgaatttatccagataaataaacatggaaattttaaaaacacaaatatgtttcttatttaaagttccctaaaactctgcatgtcatacagggatatcttatcgactgatccttgtctcttttatctacagaaatgttcttgctgcatactttga

SEQ ID NO: 71 - DNA, 699 bp, Zea mays
atggcgcaacctgaaattcctgcatttggggattgggaaaccactggaaacacactttacacacaaaagttcgaggatgcacggaagaacaagaaaacaggaattcttgcacaaccaaatgatccaaggagggatctggaacctcctcgcaagtcgcctttgcacccaacggtctacaaaactaatcctcaagaccaaggtccaaggaacccaccacatcgaccaagacctgaacttgacaaccaacgacactctgaccggcctactcaccgtgagtctgcaccacgaagacattcaaatcttcagagagaacaagggagtaatgctgggacaccaagaagcccctacagaacagctgctgggtctgcttcaccaatgcagccaaacaaccaatcaaagccaaagcacaggtcaactggaatgcagactccagagaggagggcatcttcagaggggcttggccagcatacacctggaaggagcaggatgaagccgggtggctatgagcctgaagaagtggctgtaccaccctttggcgaatgggatgatgcaaatgcagcatcaggtgaaaagtacactggtatcttcaacagggtgagggatgataggttgtcacctacctcttctgctaggcaaccgtctactactcgtagtgaagagaataaggtgcaacagaaatgttcttgctgcatactttga

SEQ ID NO: 72 - protein, 232 aa, Zea mays
MAQPEIPAFGDWETTGNTLYTQKFEDARKNKKTGILAQPNDPRRDLEPPRKSPLHPTVYKTNPQDQGPRNPPHRPRPELDNQRHSDRPTHRESAPRRHSNLQREQGSNAGTPRSPYRTAAGSASPMQPNNQSKPKHRSTGMQTPERRASSEGLGQHTPGRSRMKPGGYEPEEVAVPPFGEWDDANAASGEKYTGIFNRVRDDRLSPTSSARQPSTTRSEENKVQQKCSCCIL

SEQ ID NO: 73 - protein, 332 aa, Zea mays; identical to SEQ ID NO: 52

SEQ ID NO: 74 - protein, 232 aa, Zea mays
MAQPEIPAFEDWETTGNTPYTQKFEDVRKNKKTGIPAQTNDPRRNPEHPRKSPLHPTAYKTDPQDQGPRNPPHRPRPETDQQRHSDRPTHREPAPRRHANPHREQGSNAVAPRSPYRTAAGSASPMQSNNQSKPNHRSTATQAPERRHSSEGHSQHTPGRSRMKPGSYEPEEVAVPPFGEWDDANAASGEKYTGIFNRVRDDRLSPTSSARQPSTARREENKVQQKCSCCIL

SEQ ID NO: 75 - protein, 224 aa, Oryza sativa
MAQPDIPAFGNWDTTGNTPYTQKFENARKNKKAGISSHPNDPRRHPEPPSKSPLHPAYTPDAQGQSPMNPQHGRRQEADPHRRHSLSQQREVGGGIGSAPRSPYRMVHGSASPAQPNNPSKPKHRSSGMQTPERRASSEGHGQHTPRRSRDKQGGRGYDAPEDDVAVPPFGEWDEGNAASGEKFTGIFNRVRDDKLSPNTSTRQPDTNRSQENKVKQTCPCCIL

SEQ ID NO: 76 - protein, 220 aa, Oryza sativa
MAQPDIPAFGNWDTTGNTPYKQKFENARKNKKAGISSHPNDPRRHPEPPTKSPLHPAYTPDAQGQSPMNPQHGRRQEADPHRRHSLSQQREVGGGTGSAPRSPYRMVHGSASPAQPNNPSKPKHKSSGMQTPERRASSEGHGQHTPRRSRGKQGGRGYDAPEDDVAVPPFGEWDEGNAASGEKFTGIFNRVRDDKLSPNTSTRQPDTNRSQENKVKQAAA

SEQ ID NO: 77 - protein, 211 aa, Arabidopsis thaliana
MARSNVPKFGNWEAEENVPYTAYFDKARKTRAPGSKIMNPNDPEYNSDSQSQAPPHPPSSRTKPEQVDTVRRSREHMRSREESELKQFGDAGGSSNEAANKRQGRASQNNSYDNKSPLHKNSYDGTGKSRPKPTNLRADESPEKVTVVPKFGDWDENNPSSADGYTHIFNKVREERSSGANVSGSSRTPTHQSSRNPNNTSSCCCFGFGGK

SEQ ID NO: 78 - protein, 256 aa, Solanum tuberosum
MARPNVPKFGNWENDDNTPYTVYFEKARQTRGTGKIMNPNDPEENPDMFPNLAPPPEVAPQSKPKKQTEEPPIGRGGPARQTREHRLSKEDGEFRQYANSPARNENMGRKGANEPSHQRGRGSNSGRTGRQSIGSEHSFDKSPLHPHYQAKVNNAGRGVASPAWEGKNNNSYDSSHGTPGRSKVKQENQSDRGAAVPRFGEWDENDPQSADNYTHIFNKVREEKQGNPSGTPSRASNNTQKHNSEEKQMKWCCCPW

SEQ ID NO: 79 - protein, 85 aa, Zea mays
RPCTSTAWRSCSLKMGGAYSRTSSLGQESNRHRKFQRHWYGDRHKMRLPAACHQDSGWATVHKRENVQRLGGSLKERRMVRGRTT

SEQ ID NO: 80 - protein, 76 aa, Zea mays
RGSQRHLPELCRSTASFETVFPALLPFPKRRSHQTGGCRSDVDRRGVCTRGWVLSAARRCREHVLQRTSHAQPTRT

SEQ ID NO: 81 - protein, 12 aa, Zea mays
WPVLRSVRMHDA

SEQ ID NO: 82 - protein, 12 aa, Zea mays
PSSIVCQLSGKR

SEQ ID NO: 83 - protein, 58 aa, Zea mays
SVASYAGPKFMRHEDKSQAASAVRSHRKCAPKYLQKSEAAEGANDTPKHHGSAGRVPA

SEQ ID NO: 84 - protein, 2 aa, Zea mays
PA

SEQ ID NO: 85 - protein, 19 aa, Zea mays
AATAASRGCKPHNLAAFSV

SEQ ID NO: 86 - protein, 49 aa, Zea mays
SEASKILGAVWYHDRCNPRLDWRLEVSTVHWSAKLHKSVQSSWEHSEAA

SEQ ID NO: 87 - protein, 12 aa, Zea mays
AESSPHQGGQCE

SEQ ID NO: 88 - protein, 13 aa, Zea mays
RHPQGDREITKPC

SEQ ID NO: 89 - protein, 8 aa, Zea mays
ADWFFNTE

SEQ ID NO: 90 - protein, 45 aa, Zea mays
CCCRLEQPGGARPPSPAQPLVSKQPRESTHRLCGQESRPPRQAPP

SEQ ID NO: 91 - protein, 18 aa, Zea mays
ILKLGVHTKSRWWKSDQR

SEQ ID NO: 92 - protein, 8 aa, Zea mays
HTAGETSD

SEQ ID NO: 93 - protein, 3 aa, Zea mays
GCL

SEQ ID NO: 94 - protein, 21 aa, Zea mays
ALSTGLPRKPLTNRLLRTQAS

SEQ ID NO: 95 - protein, 9 aa, Zea mays
MDELRRDGS

SEQ ID NO: 96 - protein, 66 aa, Zea mays
VPKINKTRRLHLLRAAPRIGPSPEFRFPADQTCAIYYENWTRILLQQQCYTNRPSDAVPKAGETWI

SEQ ID NO: 97 - protein, 50 aa, Zea mays
VGWMGRMDMGQGTGAGNAKHLFSQSHKMQAQVFPPRACASNQGLERIDHI

SEQ ID NO: 98 - protein, 28 aa, Zea mays
SLQLDISCKLSPPQRSASPCQPKSRDDR

SEQ ID NO: 99 - protein, 2 aa, Zea mays
SP

SEQ ID NO: 100 - protein, 41 aa, Zea mays
TTKAFGYPMPQVECTCGFNRTAKHHIAGLCRRTVSTVLGRN

SEQ ID NO: 101 - protein, 8 aa, Zea mays
CCKARGFL

SEQ ID NO: 102 - protein, 23 aa, Zea mays
RTLQTYNPARRFRVVQDQEYPKC

SEQ ID NO: 103 - protein, 9 aa, Zea mays
SICSQRRRP

SEQ ID NO: 104 - protein, 7 aa, Zea mays
RMVCIIH

SEQ ID NO: 105 - protein, 3 aa, Zea mays
GTV

SEQ ID NO: 106 - protein, 11 aa, Zea mays
PDHKQQGVRNI

SEQ ID NO: 107 - protein, 5 aa, Zea mays
SCKVL

(19) United States
(12) Patent Application Publication
(10) Pub. No.: US 2016/0032304 A1
(43) Pub. Date: Feb. 4, 2016

(54) SLM1, A SUPPRESSOR OF LESION MIMIC PHENOTYPES

(71) Applicants: E. I. DU PONT DE NEMOURS AND COMPANY, Wilmington, DE (US); PURDUE RESEARCH FOUNDATION, West Lafayette, IN (US)

(72) Inventors: Jennifer S. Jaqueth, Wilmington, DE (US); Jiabing Ji, West Lafayette, IN (US); Gurmukh Johal, West Lafayette, IN (US); April Leonard, Wilmington, DE (US); Bailin Li, Hockessin, DE (US)

(21) Appl. No.: 14/763,729

(22) PCT Filed: Jan. 30, 2014
(86) PCT No.: PCT/US14/13772

§ 371 Date: Jul. 27, 2015

Related U.S. Application Data
(60) Provisional application No. 61/758,937, filed on Jan. 31, 2013.

(51) Int. Cl. C12N 15/82; C12Q 1/68

(52) U.S. Cl. CPC C12N 15/8241; C12Q 1/6876; C12N 15/8271; C12N 15/8273; C12N 15/8267

(57) ABSTRACT

Methods and compositions for modulating Slm1 are provided. Methods are provided for modulating the expression of Slm1 in a host plant or plant cell to modulate agronomic characteristics.

### CROSS REFERENCE TO RELATED APPLICATIONS

This application claims the benefit of U.S. Provisional Application No. 61/758937, filed Jan. 31, 2013, the entire content of which is herein incorporated by reference.

### BACKGROUND

Maize disease lesion mimics mutants (Johal et al. 1995 BioEssays 17(8):685-692) provide an excellent model to study the genetic mechanism of cell death in plants, which is still largely elusive. The phenotype of many lesion mimics varies in different genetic backgrounds, suggesting that natural variation could be harnessed to genetically dissect this phenomenal process for searching modifiers. A previous study (Penning et al. 2004 Genome 47:961-969) identified a major quantitative trait loci (designated as slm1, suppressor of lesion mimics-1) on chromosome 2 in the maize inbred line Mo20W suppressing the expression of a recessive lesion mimic mutation, les23.

### SUMMARY

The present disclosure includes methods and compositions for modulating Slm1 activity. Methods are provided for modulating the expression of Slm1 in a host plant or plant cell to modulate agronomic characteristics.

### BRIEF DESCRIPTION OF THE DRAWINGS AND SEQUENCE LISTING

The disclosure can be more fully understood from the following detailed description and the accompanying drawings and Sequence Listing which form a part of this application.

FIG. 1A-FIG. 1C show the map-based cloning of slm1 in maize and four mutant alleles at slm1. FIG. 1A: Genetic mapping of slm1 with F2 and BC populations. Genetic distance in cM is shown above the markers, and the number of recombinants below the markers. FIG. 1 B: BAC clones from WT and Mo20W in the slm1 interval were sequenced and annotated. FIG. 1C: Gene structure and molecular characterization of four mutant alleles in maize slm1.

FIG. 2: Gene structure and mutant alleles of les23 in maize.

FIG. 3 shows the physical interaction between SLM1 and LES23. Full-length protein of LES23 physically interacts with the N-terminus (1-187 aa) of SLM1 from Va35, but not with full-length SLM1 from Va35 or the putative truncated products from Mo20W.

FIG. 4 shows the fine mapping procedure of slm1.

FIG. 5 shows les23-ref homozygous plants from the BC8F2 population containing 2, 1 and 0 copies of Slm1-Mo20W (left to right, respectively).

FIG. 6A-6B show a multiple alignment of LES23 homologs from various plant species. The alignment was assembled using the Clustal W method of alignment with the default parameters for multiple alignment of GAP PENALTY=10, GAP LENGTH PENALTY=0.2, Delay Divergent Sequences=30%, DNA Transition Weight=0.5, Protein Weight Matrix=Gonnet Series, DNA Weight Matrix=IUB. A proline amino acid at position 19 is conserved across plant species but is altered into leucine in the les23-ref mutant.

FIG. 7 shows a working model of slm1 and les23 interactions.

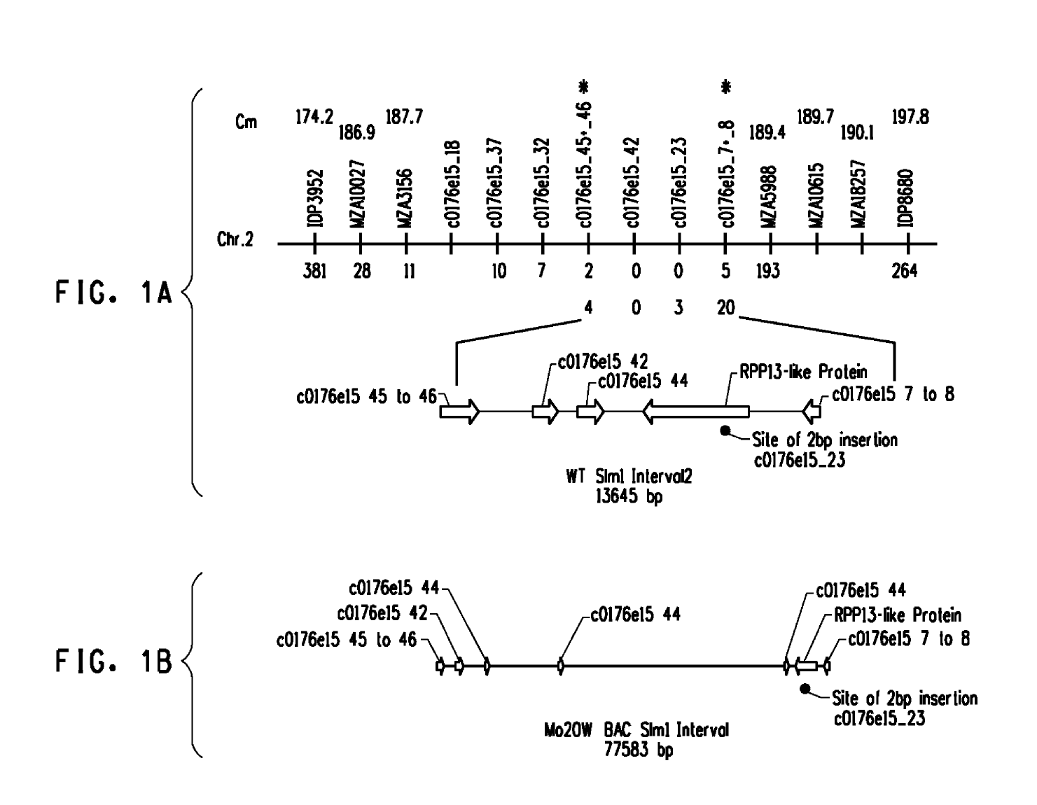

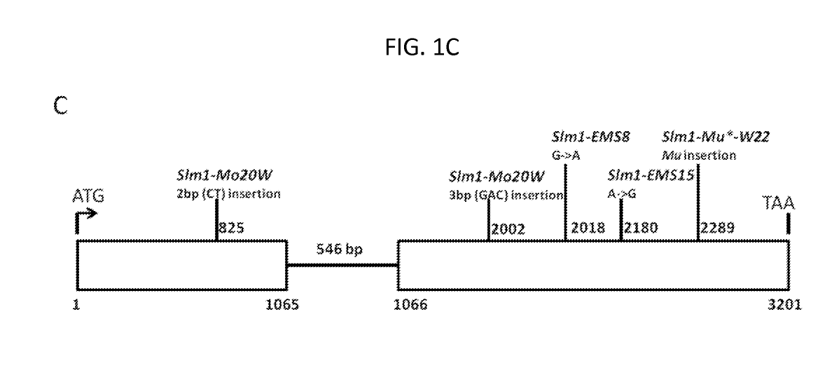

The sequence descriptions and Sequence Listing attached hereto comply with the rules governing nucleotide and/or amino acid sequence disclosures in patent applications as set forth in 37 C.F.R. §1.821-1.825.

SEQ ID NO:1-SEQ ID NO:46 are primers used in this study.

SEQ ID NO:47 is the genomic nucleotide sequence of the wild-type Slm1 locus from Va35.

SEQ ID NO:48 is the protein-coding nucleotide sequence of the wild-type Slm1 locus from Va35.

SEQ ID NO:49 is the amino acid sequence of the wild-type SLM1 protein from Va35.

SEQ ID NO:50 is the genomic nucleotide sequence of the mutant slm1 locus from Mo20W.

SEQ ID NO:51 is the nucleotide sequence of the mutant slm1 locus from Mo20W that corresponds to SEQ ID NO:48, the protein-coding region.

SEQ ID NO:52 is a translation of SEQ ID NO:51; multiple translation stop codons are present. The mutant slm1 locus encodes a truncated protein (SEQ ID NO:73).

SEQ ID NO:67 is the genomic nucleotide sequence of the wild-type Les23 locus.

SEQ ID NO:68 is the protein-coding nucleotide sequence of the wild-type Les23 locus.

SEQ ID NO:69 is the amino acid sequence of the wild-type LES23 protein.

SEQ ID NO:70 is the genomic nucleotide sequence of the mutant les23-ref locus.

SEQ ID NO:71 is the protein-coding nucleotide sequence of the mutant les23-ref locus.

SEQ ID NO:72 is the amino acid sequence of the mutant les23-ref protein.

SEQ ID NO:73 is the amino acid sequence of the truncated mutant slm1 protein encoded by SEQ ID NO:51.

SEQ ID NO:74 is the amino acid sequence of a nitrate-induced NOI protein from maize (NCBI GI No. 195622454) and corresponds to a maize LES23 paralog and is designated ZmLES23paralog.

SEQ ID NO:75 is the amino acid sequence of a rice homolog of LES23 (Os04g0379600; NCBI GI NO. 115457982) and is designated RIN4-OsJ.

SEQ ID NO:76 is the amino acid sequence of a rice homolog of LES23 (hypothetical protein Osl_{—}15598; NCBI GI NO. 218194724) and is designated RIN4-Osl.

SEQ ID NO:77 is the amino acid sequence of the Arabidopsis RIN4 protein (AT-RIN4; TAIR Accession No. 1009121715 for AT3G25070.1).

SEQ ID NO:78 is the amino acid sequence of the RIN4-like protein from Solanum tuberosum (NCBI GI NO. 565345898) and is designated StRIN4.

The Sequence Listing contains the one letter code for nucleotide sequence characters and the three letter codes for amino acids as defined in conformity with the IUPAC-IUBMB standards described in Nucleic Acids Res. 13:3021-3030 (1985) and in the Biochemical J. 219 (No. 2):345-373 (1984) which are herein incorporated by reference. The symbols and format used for nucleotide and amino acid sequence data comply with the rules set forth in 37 C.F.R. §1.822.

### DETAILED DESCRIPTION

The disclosure of each reference set forth herein is hereby incorporated by reference in its entirety.

As used herein and in the appended claims, the singular forms “a”, “an”, and “the” include plural reference unless the context clearly dictates otherwise. Thus, for example, reference to “a plant” includes a plurality of such plants, reference to “a cell” includes one or more cells and equivalents thereof known to those skilled in the art, and so forth.

As used herein:

The term “les” refers to a lesion mimic mutant. The term “les23” refers to a specific recessive lesion mimic of maize that is present on the short arm of chromosome 2 (Penning et al. 2004 Genome 47:961-969).

The term “slm1” stands for “suppressor of lesion mimics 1” and refers to a major QTL for les23 phenotype suppression that is present on chromosome 2 of maize (Penning et al. 2004 Genome 47:961-969).

The terms “monocot” and “monocotyledonous plant” are used interchangeably herein. A monocot of the current disclosure includes the Gramineae.

The terms “dicot” and “dicotyledonous plant” are used interchangeably herein. A dicot of the current disclosure includes the following families: Brassicaceae, Leguminosae, and Solanaceae.

The terms “full complement” and “full-length complement” are used interchangeably herein, and refer to a complement of a given nucleotide sequence, wherein the complement and the nucleotide sequence consist of the same number of nucleotides and are 100% complementary.

An “Expressed Sequence Tag” (“EST”) is a DNA sequence derived from a cDNA library and therefore is a sequence which has been transcribed. An EST is typically obtained by a single sequencing pass of a cDNA insert. The sequence of an entire cDNA insert is termed the “Full-Insert Sequence” (“FIS”). A “Contig” sequence is a sequence assembled from two or more sequences that can be selected from, but not limited to, the group consisting of an EST, FIS and PCR sequence. A sequence encoding an entire or functional protein is termed a “Complete Gene Sequence” (“CGS”) and can be derived from an FIS or a contig.

A “trait” refers to a physiological, morphological, biochemical, or physical characteristic of a plant or a particular plant material or cell. In some instances, this characteristic is visible to the human eye, such as seed or plant size, or can be measured by biochemical techniques, such as detecting the protein, starch, or oil content of seed or leaves, or by observation of a metabolic or physiological process, e.g. by measuring tolerance to water deprivation or particular salt or sugar concentrations, or by the observation of the expression level of a gene or genes, or by agricultural observations such as osmotic stress tolerance or yield.

“Agronomic characteristic” is a measurable parameter including but not limited to, abiotic stress tolerance, early senescence, greenness, yield, growth rate, biomass, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, fruit protein content, seed protein content, protein content in a vegetative tissue, drought tolerance, nitrogen uptake, root lodging, harvest index, stalk lodging, plant height, ear height, ear length, salt tolerance, early seedling vigor and seedling emergence under low temperature stress.

Abiotic stress may be at least one condition selected from the group consisting of: drought, water deprivation, flood, high light intensity, high temperature, low temperature, salinity, etiolation, defoliation, heavy metal toxicity, anaerobiosis, nutrient deficiency, nutrient excess, UV irradiation, atmospheric pollution (e.g., ozone) and exposure to chemicals (e.g., paraquat) that induce production of reactive oxygen species (ROS).

“Increased stress tolerance” of a plant is measured relative to a reference or control plant, and is a trait of the plant to survive under stress conditions over prolonged periods of time, without exhibiting the same degree of physiological or physical deterioration relative to the reference or control plant grown under similar stress conditions.

A plant with “increased stress tolerance” can exhibit increased tolerance to one or more different stress conditions.

“Stress tolerance activity” of a polypeptide indicates that over-expression of the polypeptide in a transgenic plant confers increased stress tolerance to the transgenic plant relative to a reference or control plant.

Increased biomass can be measured, for example, as an increase in plant height, plant total leaf area, plant fresh weight, plant dry weight or plant seed yield, as compared with control plants.

The ability to increase the biomass or size of a plant would have several important commercial applications. Crop species may be generated that produce larger cultivars, generating higher yield in, for example, plants in which the vegetative portion of the plant is useful as food, biofuel or both.

Increased leaf size may be of particular interest. Increasing leaf biomass can be used to increase production of plant-derived pharmaceutical or industrial products. An increase in total plant photosynthesis is typically achieved by increasing leaf area of the plant. Additional photosynthetic capacity may be used to increase the yield derived from particular plant tissue, including the leaves, roots, fruits or seed, or permit the growth of a plant under decreased light intensity or under high light intensity.

Modification of the biomass of another tissue, such as root tissue, may be useful to improve a plant's ability to grow under harsh environmental conditions, including drought or nutrient deprivation, because larger roots may better reach water or nutrients or take up water or nutrients.

For some ornamental plants, the ability to provide larger varieties would be highly desirable. For many plants, including fruit-bearing trees, trees that are used for lumber production, or trees and shrubs that serve as view or wind screens, increased stature provides improved benefits in the forms of greater yield or improved screening.

The growth and emergence of maize silks has a considerable importance in the determination of yield under drought (Fuad-Hassan et al. 2008 Plant Cell Environ. 31:1349-1360). When soil water deficit occurs before flowering, silk emergence out of the husks is delayed while anthesis is largely unaffected, resulting in an increased anthesis-silking interval (ASI) (Edmeades et al. 2000 Physiology and Modeling Kernel set in Maize (eds M. E.Westgate & K. Boote; CSSA (Crop Science Society of America) Special Publication No. 29. Madison, Wis.: CSSA, 43-73). Selection for reduced ASI has been used successfully to increase drought tolerance of maize (Edmeades et al. 1993 Crop Science 33: 1029-1035; Bolanos & Edmeades 1996 Field Crops Research 48:65-80; Bruce et al. 2002 J. Exp. Botany 53:13-25).

Terms used herein to describe thermal time include “growing degree days” (GDD), “growing degree units” (GDU) and “heat units” (HU).

“Transgenic” refers to any cell, cell line, callus, tissue, plant part or plant, the genome of which has been altered by the presence of a heterologous nucleic acid, such as a recombinant DNA construct, including those initial transgenic events as well as those created by sexual crosses or asexual propagation from the initial transgenic event. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

“Genome” as it applies to plant cells encompasses not only chromosomal DNA found within the nucleus, but organelle DNA found within subcellular components (e.g., mitochondrial, plastid) of the cell.

“Plant” includes reference to whole plants, plant organs, plant tissues, plant propagules, seeds and plant cells and progeny of same. Plant cells include, without limitation, cells from seeds, suspension cultures, embryos, meristematic regions, callus tissue, leaves, roots, shoots, gametophytes, sporophytes, pollen, and microspores.

“Propagule” includes all products of meiosis and mitosis able to propagate a new plant, including but not limited to, seeds, spores and parts of a plant that serve as a means of vegetative reproduction, such as corms, tubers, offsets, or runners. Propagule also includes grafts where one portion of a plant is grafted to another portion of a different plant (even one of a different species) to create a living organism. Propagule also includes all plants and seeds produced by cloning or by bringing together meiotic products, or allowing meiotic products to come together to form an embryo or fertilized egg (naturally or with human intervention).

“Progeny” comprises any subsequent generation of a plant.

“Transgenic plant” includes reference to a plant which comprises within its genome a heterologous polynucleotide. For example, the heterologous polynucleotide is stably integrated within the genome such that the polynucleotide is passed on to successive generations. The heterologous polynucleotide may be integrated into the genome alone or as part of a recombinant DNA construct.

The commercial development of genetically improved germplasm has also advanced to the stage of introducing multiple traits into crop plants, often referred to as a gene stacking approach. In this approach, multiple genes conferring different characteristics of interest can be introduced into a plant. Gene stacking can be accomplished by many means including but not limited to co-transformation, retransformation, and crossing lines with different transgenes.

“Transgenic plant” also includes reference to plants which comprise more than one heterologous polynucleotide within their genome. Each heterologous polynucleotide may confer a different trait to the transgenic plant.

“Heterologous” with respect to sequence means a sequence that originates from a foreign species, or, if from the same species, is substantially modified from its native form in composition and/or genomic locus by deliberate human intervention.

“Polynucleotide”, “nucleic acid sequence”, “nucleotide sequence”, or “nucleic acid fragment” are used interchangeably and is a polymer of RNA or DNA that is single- or double-stranded, optionally containing synthetic, non-natural or altered nucleotide bases. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

“Polypeptide”, “peptide”, “amino acid sequence” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The terms “polypeptide”, “peptide”, “amino acid sequence”, and “protein” are also inclusive of modifications including, but not limited to, glycosylation, lipid attachment, sulfation, gamma-carboxylation of glutamic acid residues, hydroxylation and ADP-ribosylation.

“Messenger RNA (mRNA)” refers to the RNA that is without introns and that can be translated into protein by the cell.

“cDNA” refers to a DNA that is complementary to and synthesized from a mRNA template using the enzyme reverse transcriptase. The cDNA can be single-stranded or converted into the double-stranded form using the Klenow fragment of DNA polymerase I.

“Coding region” refers to the portion of a messenger RNA (or the corresponding portion of another nucleic acid molecule such as a DNA molecule) which encodes a protein or polypeptide. “Non-coding region” refers to all portions of a messenger RNA or other nucleic acid molecule that are not a coding region, including but not limited to, for example, the promoter region, 5′ untranslated region (“UTR”), 3′ UTR, intron and terminator. The terms “coding region” and “coding sequence” are used interchangeably herein. The terms “non-coding region” and “non-coding sequence” are used interchangeably herein.

“Mature” protein refers to a post-translationally processed polypeptide; i.e., one from which any pre- or pro-peptides present in the primary translation product have been removed.

“Precursor” protein refers to the primary product of translation of mRNA; i.e., with pre- and pro-peptides still present. Pre- and pro-peptides may be and are not limited to intracellular localization signals.

“Isolated” refers to materials, such as nucleic acid molecules and/or proteins, which are substantially free or otherwise removed from components that normally accompany or interact with the materials in a naturally occurring environment. Isolated polynucleotides may be purified from a host cell in which they naturally occur. Conventional nucleic acid purification methods known to skilled artisans may be used to obtain isolated polynucleotides. The term also embraces recombinant polynucleotides and chemically synthesized polynucleotides.

“Recombinant” refers to an artificial combination of two otherwise separated segments of sequence, e.g., by chemical synthesis or by the manipulation of isolated segments of nucleic acids by genetic engineering techniques. “Recombinant” also includes reference to a cell or vector, that has been modified by the introduction of a heterologous nucleic acid or a cell derived from a cell so modified, but does not encompass the alteration of the cell or vector by naturally occurring events (e.g., spontaneous mutation, natural transformation/transduction/transposition) such as those occurring without deliberate human intervention.

“Recombinant DNA construct” refers to a combination of nucleic acid fragments that are not normally found together in nature. Accordingly, a recombinant DNA construct may comprise regulatory sequences and coding sequences that are derived from different sources, or regulatory sequences and coding sequences derived from the same source, but arranged in a manner different than that normally found in nature. The terms “recombinant DNA construct” and “recombinant construct” are used interchangeably herein.

The terms “entry clone” and “entry vector” are used interchangeably herein.

“Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences. The terms “regulatory sequence” and “regulatory element” are used interchangeably herein.

“Promoter” refers to a nucleic acid fragment capable of controlling transcription of another nucleic acid fragment.

“Promoter functional in a plant” is a promoter capable of controlling transcription in plant cells whether or not its origin is from a plant cell.

“Tissue-specific promoter” and “tissue-preferred promoter” are used interchangeably, and refer to a promoter that is expressed predominantly but not necessarily exclusively in one tissue or organ, but that may also be expressed in one specific cell.

“Developmentally regulated promoter” refers to a promoter whose activity is determined by developmental events.

“Operably linked” refers to the association of nucleic acid fragments in a single fragment so that the function of one is regulated by the other. For example, a promoter is operably linked with a nucleic acid fragment when it is capable of regulating the transcription of that nucleic acid fragment.

“Expression” refers to the production of a functional product. For example, expression of a nucleic acid fragment may refer to transcription of the nucleic acid fragment (e.g., transcription resulting in mRNA or functional RNA) and/or translation of mRNA into a precursor or mature protein.

“Phenotype” means the detectable characteristics of a cell or organism.

“Introduced” in the context of inserting a nucleic acid fragment (e.g., a recombinant DNA construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

A “transformed cell” is any cell into which a nucleic acid fragment (e.g., a recombinant DNA construct) has been introduced.

“Transformation” as used herein refers to both stable transformation and transient transformation.

“Stable transformation” refers to the introduction of a nucleic acid fragment into a genome of a host organism resulting in genetically stable inheritance. Once stably transformed, the nucleic acid fragment is stably integrated in the genome of the host organism and any subsequent generation.

“Transient transformation” refers to the introduction of a nucleic acid fragment into the nucleus, or DNA-containing organelle, of a host organism resulting in gene expression without genetically stable inheritance.

“Allele” is one of several alternative forms of a gene occupying a given locus on a chromosome. When the alleles present at a given locus on a pair of homologous chromosomes in a diploid plant are the same that plant is homozygous at that locus. If the alleles present at a given locus on a pair of homologous chromosomes in a diploid plant differ that plant is heterozygous at that locus. If a transgene is present on one of a pair of homologous chromosomes in a diploid plant that plant is hemizygous at that locus.

A “chloroplast transit peptide” is an amino acid sequence which is translated in conjunction with a protein and directs the protein to the chloroplast or other plastid types present in the cell in which the protein is made (Lee et al. (2008) Plant Cell 20:1603-1622). The terms “chloroplast transit peptide” and “plastid transit peptide” are used interchangeably herein. “Chloroplast transit sequence” refers to a nucleotide sequence that encodes a chloroplast transit peptide. A “signal peptide” is an amino acid sequence which is translated in conjunction with a protein and directs the protein to the secretory system (Chrispeels (1991) Ann. Rev. Plant Phys. Plant Mol. Biol. 42:21-53). If the protein is to be directed to a vacuole, a vacuolar targeting signal (supra) can further be added, or if to the endoplasmic reticulum, an endoplasmic reticulum retention signal (supra) may be added. If the protein is to be directed to the nucleus, any signal peptide present should be removed and instead a nuclear localization signal included (Raikhel (1992) Plant Phys. 100:1627-1632). A “mitochondrial signal peptide” is an amino acid sequence which directs a precursor protein into the mitochondria (Zhang and Glaser (2002) Trends Plant Sci 7:14-21).

Sequence alignments and percent identity calculations may be determined using a variety of comparison methods designed to detect homologous sequences including, but not limited to, the Megalign® program of the LASERGENE® bioinformatics computing suite (DNASTAR® Inc., Madison, Wis.). Unless stated otherwise, multiple alignment of the sequences provided herein were performed using the Clustal V method of alignment (Higgins and Sharp (1989) CABIOS. 5:151-153) with the default parameters (GAP PENALTY=10, GAP LENGTH PENALTY=10). Default parameters for pairwise alignments and calculation of percent identity of protein sequences using the Clustal V method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences, using the Clustal V program, it is possible to obtain “percent identity” and “divergence” values by viewing the “sequence distances” table on the same program; unless stated otherwise, percent identities and divergences provided and claimed herein were calculated in this manner.

Alternatively, the Clustal W method of alignment may be used. The Clustal W method of alignment (described by Higgins and Sharp, CABIOS. 5:151-153 (1989); Higgins, D. G. et al., Comput. Appl. Biosci. 8:189-191 (1992)) can be found in the MegAlign™ v6.1 program of the LASERGENE® bioinformatics computing suite (DNASTAR® Inc., Madison, Wis.). Default parameters for multiple alignment correspond to GAP PENALTY=10, GAP LENGTH PENALTY=0.2, Delay Divergent Sequences=30%, DNA Transition Weight=0.5, Protein Weight Matrix=Gonnet Series, DNA Weight Matrix=IUB. For pairwise alignments the default parameters are Alignment=Slow-Accurate, Gap Penalty=10.0, Gap Length=0.10, Protein Weight Matrix=Gonnet 250 and DNA Weight Matrix=IUB. After alignment of the sequences using the Clustal W program, it is possible to obtain “percent identity” and “divergence” values by viewing the “sequence distances” table in the same program.

The term “under stringent conditions” means that two sequences hybridize under moderately or highly stringent conditions. More specifically, moderately stringent conditions can be readily determined by those having ordinary skill in the art, e.g., depending on the length of DNA. The basic conditions are set forth by Sambrook et al., Molecular Cloning: A Laboratory Manual, third edition, chapters 6 and 7, Cold Spring Harbor Laboratory Press, 2001 and include the use of a prewashing solution for nitrocellulose filters 5×SSC, 0.5% SDS, 1.0 mM EDTA (pH 8.0), hybridization conditions of about 50% formamide, 2×SSC to 6×SSC at about 40-50° C. (or other similar hybridization solutions, such as Stark's solution, in about 50% formamide at about 42° C.) and washing conditions of, for example, about 40-60° C., 0.5-6×SSC, 0.1% SDS. Preferably, moderately stringent conditions include hybridization (and washing) at about 50° C. and 6×SSC. Highly stringent conditions can also be readily determined by those skilled in the art, e.g., depending on the length of DNA.

Generally, such conditions include hybridization and/or washing at higher temperature and/or lower salt concentration (such as hybridization at about 65° C., 6×SSC to 0.2×SSC, preferably 6×SSC, more preferably 2×SSC, most preferably 0.2×SSC), compared to the moderately stringent conditions. For example, highly stringent conditions may include hybridization as defined above, and washing at approximately 65-68° C., 0.2×SSC, 0.1% SDS. SSPE (1×SSPE is 0.15 M NaCl, 10 mM NaH2PO4, and 1.25 mM EDTA, pH 7.4) can be substituted for SSC (1×SSC is 0.15 M NaCl and 15 mM sodium citrate) in the hybridization and washing buffers; washing is performed for 15 minutes after hybridization is completed.

It is also possible to use a commercially available hybridization kit which uses no radioactive substance as a probe. Specific examples include hybridization with an ECL direct labeling & detection system (Amersham). Stringent conditions include, for example, hybridization at 42° C. for 4 hours using the hybridization buffer included in the kit, which is supplemented with 5% (w/v) Blocking reagent and 0.5 M NaCl, and washing twice in 0.4% SDS, 0.5×SSC at 55° C. for 20 minutes and once in 2×SSC at room temperature for 5 minutes.

Standard recombinant DNA and molecular cloning techniques used herein are well known in the art and are described more fully in Sambrook, J., Fritsch, E.F. and Maniatis, T. Molecular Cloning: A Laboratory Manual; Cold Spring Harbor Laboratory Press: Cold Spring Harbor, 1989 (hereinafter “Sambrook”).

Slm1 was discovered as a QTL in a natural enhancer/suppressor screen named “MAGIC”, for “Mutant-Assisted Gene Identification and Characterization”. The reporter mutant phenotype used in this study was provided by les23, a recessive lesion mimic mutation that results in precocious leaf death and ear abortion. To understand how Slm1 may suppress les23, we cloned and confirmed the gene responsible for Slm1 by a combination of approaches involving positional cloning, transposon tagging with Mutator (Mu) and directed mutagenesis with EMS. The results revealed that a 2-base pair insertion in the 5′ half of an NBS-LRR R gene, which results in a frame-shift and truncated protein, was responsible for the les23-suppressing phenotype of Slm1. To determine how a defective R gene encoding a truncated protein could act as a potent suppressor of les23, we cloned the les23 gene. A missense mutation leading to single amino acid substitution in a homolog of the Arabidopsis RIN4 gene was found to cause the les23 mutation. Originally identified as an interactor of the R protein RPM1, RIN4 has emerged as a key component of the guard mechanism of plant innate immune responses. Absence or degradation of RIN4 leads to a robust hypersensitive cell death response mediated by the R protein RPS2. In this regard, the slm1 gene seems to be a functional equivalent of Arabidopsis RPS2, triggering les23 lesions when the maize RIN4 is mutated. However, if slm1 is dysfunctional, as it is in the les23-suppressing QTL Slm1, no cell death is initiated whether the maize Rin4 (ZmRin4) is defective or not. Consistent with this model, the intact SLM1 protein can physically interact with the wild-type ZmRIN4 protein but not with the mutant ZmRIN4. Thus, what appeared to be a gain-of-function QTL genetically, is in fact a loss-of-function allele of a maize R gene guard.

An insecticidal protein system discovered in Bacillus thuringiensis has been disclosed in WO 97/40162. This system comprises two proteins; one of approximately 15 kDa and the other of about 45 kDa. See also U.S. Pat. Nos. 6,083,499 and 6,127,180. These proteins have been assigned the Cry designations of Cry34 and Cr35, respectively. The Cry34 and Cry35 classes function as binary toxins showing activity on the western corn rootworm, Diabrotica virgifera virgifera LeConte (Schnepf et al. 2005 Applied and Environmental Microbiology 71:1765-1774).

In the transgenic corn line HXRW, two separate parasporal crystal proteins are expressed, Cry34 and Cry35, with respective molecular weights of 14 kDa and 44 kDa. Both insecticidal crystal proteins (ICPs) are required to provide commercial levels of activity on western (Diabrotica virgifiera virgifera), northern (Diabrotica berberi) and Mexican corn rootworm (Diabrotica virgifera zeae, CRW) larvae.

Cry34/35 transgenes present in HXRW have been associated with a distinct early senescence (leaf fire) phenotype in certain inbred backgrounds. The slm1 allele was examined as a method to reduce the amount of early senescence in inbred conversions with the HXRW transgene. Near-Isogenic Lines (NILs) were created by backcrossing the slm1 donor allele from Mo20W into three recurrent parents containing the HXRW transgene: InbredA-HXRW, InbredB-HXRW, and InbredC-HXRW. The lines were backrossed three generations and then selfed twice, to create BC3S2 NILs both with the Mo20W slm1 allele and without the Mo20W slm1 allele. The InbredA inbred background was previously identified as showing a severe leaf fire phenotype in the presence of the HXRW transgene, and the InbredB and InbredC inbreds showed less severe leaf firing.

Turning now to the embodiments:

Embodiments include isolated polynucleotides and polypeptides, recombinant DNA constructs (including suppression DNA constructs), compositions (such as plants or seeds) comprising these recombinant DNA constructs, and methods utilizing these recombinant DNA constructs.

In another embodiment, a recombinant DNA construct comprises a polynucleotide operably linked to at least one regulatory sequence (e.g., a promoter functional in a plant), wherein said polynucleotide comprises all or a fragment of SEQ ID NO:47, 48, 50 or 51, or encodes all or a fragment of SEQ ID NO:49, 52 or 73.

“Suppression DNA construct” is a recombinant DNA construct which when transformed or stably integrated into the genome of the plant, results in “silencing” of a target gene in the plant. The target gene may be endogenous or transgenic to the plant. “Silencing,” as used herein with respect to the target gene, refers generally to the suppression of levels of mRNA or protein/enzyme expressed by the target gene, and/or the level of the enzyme activity or protein functionality. The terms “suppression”, “suppressing” and “silencing”, used interchangeably herein, include lowering, reducing, declining, decreasing, inhibiting, eliminating or preventing. “Silencing” or “gene silencing” does not specify mechanism and is inclusive, and not limited to, anti-sense, cosuppression, viral-suppression, hairpin suppression, stem-loop suppression, RNAi-based approaches, and small RNA-based approaches.

A suppression DNA construct may comprise a region derived from a target gene of interest and may comprise all or part of the nucleic acid sequence of the sense strand (or antisense strand) of the target gene of interest. Depending upon the approach to be utilized, the region may be 100% identical or less than 100% identical (e.g., at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical) to all or part of the sense strand (or antisense strand) of the gene of interest.

A suppression DNA construct may comprise 100, 200, 300, 400, 500, 600, 700, 800, 900 or 1000 contiguous nucleotides of the sense strand (or antisense strand) of the gene of interest, and combinations thereof.

Suppression DNA constructs are well-known in the art, are readily constructed once the target gene of interest is selected, and include, without limitation, cosuppression constructs, antisense constructs, viral-suppression constructs, hairpin suppression constructs, stem-loop suppression constructs, double-stranded RNA-producing constructs, and more generally, RNAi (RNA interference) constructs and small RNA constructs such as siRNA (short interfering RNA) constructs and miRNA (microRNA) constructs.

Suppression of gene expression may also be achieved by use of artificial miRNA precursors, ribozyme constructs and gene disruption. A modified plant miRNA precursor may be used, wherein the precursor has been modified to replace the miRNA encoding region with a sequence designed to produce a miRNA directed to the nucleotide sequence of interest. Gene disruption may be achieved by use of transposable elements or by use of chemical agents that cause site-specific mutations.

“Antisense inhibition” refers to the production of antisense RNA transcripts capable of suppressing the expression of the target gene or gene product. “Antisense RNA” refers to an RNA transcript that is complementary to all or part of a target primary transcript or mRNA and that blocks the expression of a target isolated nucleic acid fragment (U.S. Pat. No. 5,107,065). The complementarity of an antisense RNA may be with any part of the specific gene transcript, i.e., at the 5′ non-coding sequence, 3′ non-coding sequence, introns, or the coding sequence.

“Cosuppression” refers to the production of sense RNA transcripts capable of suppressing the expression of the target gene or gene product. “Sense” RNA refers to RNA transcript that includes the mRNA and can be translated into protein within a cell or in vitro. Cosuppression constructs in plants have been previously designed by focusing on overexpression of a nucleic acid sequence having homology to a native mRNA, in the sense orientation, which results in the reduction of all RNA having homology to the overexpressed sequence (see Vaucheret et al., Plant J. 16:651-659 (1998); and Gura, Nature 404:804-808 (2000)).

Another variation describes the use of plant viral sequences to direct the suppression of proximal mRNA encoding sequences (PCT Publication No. WO 98/36083 published on Aug. 20, 1998).

RNA interference refers to the process of sequence-specific post-transcriptional gene silencing in animals mediated by short interfering RNAs (siRNAs) (Fire et al., Nature 391:806 (1998)). The corresponding process in plants is commonly referred to as post-transcriptional gene silencing (PTGS) or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla (Fire et al., Trends Genet. 15:358 (1999)).

Small RNAs play an important role in controlling gene expression. Regulation of many developmental processes, including flowering, is controlled by small RNAs. It is now possible to engineer changes in gene expression of plant genes by using transgenic constructs which produce small RNAs in the plant.

Small RNAs appear to function by base-pairing to complementary RNA or DNA target sequences. When bound to RNA, small RNAs trigger either RNA cleavage or translational inhibition of the target sequence. When bound to DNA target sequences, it is thought that small RNAs can mediate DNA methylation of the target sequence. The consequence of these events, regardless of the specific mechanism, is that gene expression is inhibited.

MicroRNAs (miRNAs) are noncoding RNAs of about 19 to about 24 nucleotides (nt) in length that have been identified in both animals and plants (Lagos-Quintana et al., Science 294:853-858 (2001), Lagos-Quintana et al., Curr. Biol. 12:735-739 (2002); Lau et al., Science 294:858-862 (2001); Lee and Ambros, Science 294:862-864 (2001); Llave et al., Plant Cell 14:1605-1619 (2002); Mourelatos et al., Genes Dev. 16:720-728 (2002); Park et al., Curr. Biol. 12:1484-1495 (2002); Reinhart et al., Genes. Dev. 16:1616-1626 (2002)). They are processed from longer precursor transcripts that range in size from approximately 70 to 200 nt, and these precursor transcripts have the ability to form stable hairpin structures.

MicroRNAs (miRNAs) appear to regulate target genes by binding to complementary sequences located in the transcripts produced by these genes. It seems likely that miRNAs can enter at least two pathways of target gene regulation: (1) translational inhibition; and (2) RNA cleavage. MicroRNAs entering the RNA cleavage pathway are analogous to the 21-25 nt short interfering RNAs (siRNAs) generated during RNA interference (RNAi) in animals and posttranscriptional gene silencing (PTGS) in plants, and likely are incorporated into an RNA-induced silencing complex (RISC) that is similar or identical to that seen for RNAi.

The terms “miRNA-star sequence” and “miRNA*sequence” are used interchangeably herein and they refer to a sequence in the miRNA precursor that is highly complementary to the miRNA sequence. The miRNA and miRNA* sequences form part of the stem region of the miRNA precursor hairpin structure.

In one embodiment, there is provided a method for the suppression of a target sequence comprising introducing into a cell a nucleic acid construct encoding a miRNA substantially complementary to the target. In some embodiments the miRNA comprises about 19, 20, 21, 22, 23, 24 or 25 nucleotides. In some embodiments the miRNA comprises 21 nucleotides. In some embodiments the nucleic acid construct encodes the miRNA. In some embodiments the nucleic acid construct encodes a polynucleotide precursor which may form a double-stranded RNA, or hairpin structure comprising the miRNA.

In some embodiments, the nucleic acid construct comprises a modified endogenous plant miRNA precursor, wherein the precursor has been modified to replace the endogenous miRNA encoding region with a sequence designed to produce a miRNA directed to the target sequence. The plant miRNA precursor may be full-length of may comprise a fragment of the full-length precursor. In some embodiments, the endogenous plant miRNA precursor is from a dicot or a monocot. In some embodiments the endogenous miRNA precursor is from Arabidopsis, tomato, maize, soybean, sunflower, sorghum, canola, wheat, alfalfa, cotton, rice, barley, millet, sugar cane or switchgrass.

In some embodiments, the miRNA template, (i.e. the polynucleotide encoding the miRNA), and thereby the miRNA, may comprise some mismatches relative to the target sequence. In some embodiments the miRNA template has >1 nucleotide mismatch as compared to the target sequence, for example, the miRNA template can have 1, 2, 3, 4, 5, or more mismatches as compared to the target sequence. This degree of mismatch may also be described by determining the percent identity of the miRNA template to the complement of the target sequence. For example, the miRNA template may have a percent identity including about at least 70%, 75%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% as compared to the complement of the target sequence.

In some embodiments, the miRNA template, (i.e. the polynucleotide encoding the miRNA) and thereby the miRNA, may comprise some mismatches relative to the miRNA-star sequence. In some embodiments the miRNA template has >1 nucleotide mismatch as compared to the miRNA-star sequence, for example, the miRNA template can have 1, 2, 3, 4, 5, or more mismatches as compared to the miRNA-star sequence. This degree of mismatch may also be described by determining the percent identity of the miRNA template to the complement of the miRNA-star sequence. For example, the miRNA template may have a percent identity including about at least 70%, 75%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% as compared to the complement of the miRNA-star sequence.

Regulatory Sequences:

A recombinant DNA construct (including a suppression DNA construct) of the present disclosure may comprise at least one regulatory sequence.

A regulatory sequence may be a promoter.

A number of promoters can be used in recombinant DNA constructs of the present disclosure. The promoters can be selected based on the desired outcome, and may include constitutive, tissue-specific, inducible, or other promoters for expression in the host organism.

Promoters that cause a gene to be expressed in most cell types at most times are commonly referred to as “constitutive promoters”.

High level, constitutive expression of the candidate gene under control of the 35S or UBI promoter may have pleiotropic effects, although candidate gene efficacy may be estimated when driven by a constitutive promoter. Use of tissue-specific and/or stress-specific promoters may eliminate undesirable effects but retain the ability to enhance drought tolerance. This effect has been observed in Arabidopsis (Kasuga et al. (1999) Nature Biotechnol. 17:287-91).

Suitable constitutive promoters for use in a plant host cell include, for example, the core promoter of the Rsyn7 promoter and other constitutive promoters disclosed in WO 99/43838 and U.S. Pat. No. 6,072,050; the core CaMV 35S promoter (Odell et al., Nature 313:810-812 (1985)); rice actin (McElroy et al., Plant Cell 2:163-171 (1990)); ubiquitin (Christensen et al., Plant Mol. Biol. 12:619-632 (1989) and Christensen et al., Plant Mol. Biol. 18:675-689 (1992)); pEMU (Last et al., Theor. Appl. Genet. 81:581-588 (1991)); MAS (Velten et al., EMBO J. 3:2723-2730 (1984)); ALS promoter (U.S. Pat. No. 5,659,026), the constitutive synthetic core promoter SCP1 (International Publication No. 03/033651) and the like. Other constitutive promoters include, for example, those discussed in U.S. Pat. Nos. 5,608,149; 5,608,144; 5,604,121; 5,569,597; 5,466,785; 5,399,680; 5,268,463; 5,608,142; and 6,177,611.

In choosing a promoter to use in the methods of the disclosure, it may be desirable to use a tissue-specific or developmentally regulated promoter.

A tissue-specific or developmentally regulated promoter is a DNA sequence which regulates the expression of a DNA sequence selectively in the cells/tissues of a plant critical to tassel development, seed set, or both, and limits the expression of such a DNA sequence to the period of tassel development or seed maturation in the plant. Any identifiable promoter may be used in the methods of the present disclosure which causes the desired temporal and spatial expression.

Promoters which are seed or embryo-specific and may be useful in the disclosure include soybean Kunitz trypsin inhibitor (Kti3, Jofuku and Goldberg, Plant Cell 1:1079-1093 (1989)), patatin (potato tubers) (Rocha-Sosa, M., et al. (1989) EMBO J. 8:23-29), convicilin, vicilin, and legumin (pea cotyledons) (Rerie, W. G., et al. (1991) Mol. Gen. Genet. 259:149-157; Newbigin, E. J., et al. (1990) Planta 180:461-470; Higgins, T. J. V., et al. (1988) Plant. Mol. Biol. 11:683-695), zein (maize endosperm) (Schemthaner, J. P., et al. (1988) EMBO J. 7:1249-1255), phaseolin (bean cotyledon) (Segupta-Gopalan, C., et al. (1985) Proc. Natl. Acad. Sci. U.S.A. 82:3320-3324), phytohemagglutinin (bean cotyledon) (Voelker, T. et al. (1987) EMBO J. 6:3571-3577), B-conglycinin and glycinin (soybean cotyledon) (Chen, Z-L, et al. (1988) EMBO J. 7:297-302), glutelin (rice endosperm), hordein (barley endosperm) (Marris, C., et al. (1988) Plant Mol. Biol. 10:359-366), glutenin and gliadin (wheat endosperm) (Colot, V., et al. (1987) EMBO J. 6:3559-3564), and sporamin (sweet potato tuberous root) (Hattori, T., et al. (1990) Plant Mol. Biol. 14:595-604). Promoters of seed-specific genes operably linked to heterologous coding regions in chimeric gene constructions maintain their temporal and spatial expression pattern in transgenic plants. Such examples include Arabidopsis thaliana 2S seed storage protein gene promoter to express enkephalin peptides in Arabidopsis and Brassica napus seeds (Vanderkerckhove et al., Bio/Technology 7:L929-932 (1989)), bean lectin and bean beta-phaseolin promoters to express luciferase (Riggs et al., Plant Sci. 63:47-57 (1989)), and wheat glutenin promoters to express chloramphenicol acetyl transferase (Colot et al., EMBO J 6:3559-3564 (1987)).

Inducible promoters selectively express an operably linked DNA sequence in response to the presence of an endogenous or exogenous stimulus, for example by chemical compounds (chemical inducers) or in response to environmental, hormonal, chemical, and/or developmental signals. Inducible or regulated promoters include, for example, promoters regulated by light, heat, stress, flooding or drought, phytohormones, wounding, or chemicals such as ethanol, jasmonate, salicylic acid, or safeners.

Promoters for use in the current disclosure include the following: 1) the stress-inducible RD29A promoter (Kasuga et al. (1999) Nature Biotechnol. 17:287-91); 2) the barley promoter, B22E; expression of B22E is specific to the pedicel in developing maize kernels (“Primary Structure of a Novel Barley Gene Differentially Expressed in Immature Aleurone Layers”. Klemsdal, S. S. et al., Mol. Gen. Genet. 228(1/2):9-16 (1991)); and 3) maize promoter, Zag2 (“Identification and molecular characterization of ZAG 1, the maize homolog of the Arabidopsis floral homeotic gene AGAMOUS”, Schmidt, R. J. et al., Plant Cell 5(7):729-737 (1993); “Structural characterization, chromosomal localization and phylogenetic evaluation of two pairs of AGAMOUS-like MADS-box genes from maize”, Theissen et al. Gene 156(2):155-166 (1995); NCBI GenBank Accession No. X80206)). Zag2 transcripts can be detected 5 days prior to pollination to 7 to 8 days after pollination (“DAP”), and directs expression in the carpel of developing female inflorescences and Ciml which is specific to the nucleus of developing maize kernels. Ciml transcript is detected 4 to 5 days before pollination to 6 to 8 DAP. Other useful promoters include any promoter which can be derived from a gene whose expression is maternally associated with developing female florets.

Additional promoters for regulating the expression of the nucleotide sequences of the present disclosure in plants are stalk-specific promoters. Such stalk-specific promoters include the alfalfa S2A promoter (GenBank Accession No. EF030816; Abrahams et al., Plant Mol. Biol. 27:513-528 (1995)) and S2B promoter (GenBank Accession No. EF030817) and the like, herein incorporated by reference.

Promoters may be derived in their entirety from a native gene, or be composed of different elements derived from different promoters found in nature, or even comprise synthetic DNA segments.

Promoters for use in the current disclosure may include: RIP2, mLIP15, ZmCOR1, Rab17, CaMV 35S, RD29A, B22E, Zag2, SAM synthetase, ubiquitin, CaMV 19S, nos, Adh, sucrose synthase, R-allele, the vascular tissue preferred promoters S2A (Genbank accession number EF030816) and S2B (Genbank accession number EF030817), and the constitutive promoter GOS2 from Zea mays. Other promoters include root preferred promoters, such as the maize NAS2 promoter, the maize Cyclo promoter (US 2006/0156439, published Jul. 13, 2006), the maize ROOTMET2 promoter (WO05063998, published Jul. 14, 2005), the CR1BIO promoter (WO06055487, published May 26, 2006), the CRWAQ81 (WO05035770, published Apr. 21, 2005) and the maize ZRP2.47 promoter (NCBI accession number: U38790; GI No. 1063664),

Recombinant DNA constructs of the present disclosure may also include other regulatory sequences, including but not limited to, translation leader sequences, introns, and polyadenylation recognition sequences. In another embodiment of the present disclosure, a recombinant DNA construct of the present disclosure further comprises an enhancer or silencer.

An intron sequence can be added to the 5′ untranslated region, the protein-coding region or the 3′ untranslated region to increase the amount of the mature message that accumulates in the cytosol. Inclusion of a spliceable intron in the transcription unit in both plant and animal expression constructs has been shown to increase gene expression at both the mRNA and protein levels up to 1000-fold. Buchman and Berg, Mol. Cell Biol. 8:4395-4405 (1988); Callis et al., Genes Dev. 1:1183-1200 (1987).

Examples of suitable plants for the isolation of genes and regulatory sequences and for compositions and methods of the present disclosure would include but are not limited to alfalfa, apple, apricot, Arabidopsis, artichoke, arugula, asparagus, avocado, banana, barley, beans, beet, blackberry, blueberry, broccoli, brussels sprouts, cabbage, canola, cantaloupe, carrot, cassava, castorbean, cauliflower, celery, cherry, chicory, cilantro, citrus, clementines, clover, coconut, coffee, corn, cotton, cranberry, cucumber, Douglas fir, eggplant, endive, escarole, eucalyptus, fennel, figs, garlic, gourd, grape, grapefruit, honey dew, jicama, kiwifruit, lettuce, leeks, lemon, lime, Loblolly pine, linseed, mango, melon, mushroom, nectarine, nut, oat, oil palm, oil seed rape, okra, olive, onion, orange, an ornamental plant, palm, papaya, parsley, parsnip, pea, peach, peanut, pear, pepper, persimmon, pine, pineapple, plantain, plum, pomegranate, poplar, potato, pumpkin, quince, radiata pine, radicchio, radish, rapeseed, raspberry, rice, rye, sorghum, Southern pine, soybean, spinach, squash, strawberry, sugarbeet, sugarcane, sunflower, sweet potato, sweetgum, switchgrass, tangerine, tea, tobacco, tomato, triticale, turf, turnip, a vine, watermelon, wheat, yams, and zucchini.

A composition of the present disclosure includes a transgenic microorganism, cell, plant, and seed comprising the recombinant DNA construct. The cell may be eukaryotic, e.g., a yeast, insect or plant cell, or prokaryotic, e.g., a bacterial cell.

A composition of the present disclosure is a plant comprising in its genome any of the recombinant DNA constructs (including any of the suppression DNA constructs) of the present disclosure (such as any of the constructs discussed above). Compositions also include any progeny of the plant, and any seed obtained from the plant or its progeny, wherein the progeny or seed comprises within its genome the recombinant DNA construct (or suppression DNA construct). Progeny includes subsequent generations obtained by self-pollination or out-crossing of a plant. Progeny also includes hybrids and inbreds.

In hybrid seed propagated crops, mature transgenic plants can be self-pollinated to produce a homozygous inbred plant. The inbred plant produces seed containing the newly introduced recombinant DNA construct (or suppression DNA construct). These seeds can be grown to produce plants that would exhibit an altered agronomic characteristic (e.g., an increased agronomic characteristic optionally under water limiting conditions), or used in a breeding program to produce hybrid seed, which can be grown to produce plants that would exhibit such an altered agronomic characteristic. The seeds may be maize seeds.

The plant may be a monocotyledonous or dicotyledonous plant, for example, a maize or soybean plant. The plant may also be sunflower, sorghum, canola, wheat, alfalfa, cotton, rice, barley, millet, sugar cane or switchgrass. The plant may be a hybrid plant or an inbred plant.

The recombinant DNA construct may be stably integrated into the genome of the plant.

In any of the embodiments described herein, the recombinant DNA construct (or suppression DNA construct) may comprise at least a promoter functional in a plant as a regulatory sequence.

In any of the embodiments described herein or any other embodiments of the present disclosure, the alteration of at least one agronomic characteristic is either an increase or decrease.

In any of the embodiments described herein, the at least one agronomic characteristic may be selected from the group consisting of: abiotic stress tolerance, early senescence, greenness, yield, growth rate, biomass, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, fruit protein content, seed protein content, protein content in a vegetative tissue, drought tolerance, nitrogen uptake, root lodging, harvest index, stalk lodging, plant height, ear height, ear length, salt tolerance, early seedling vigor and seedling emergence under low temperature stress. For example, the alteration of at least one agronomic characteristic may be an increase in yield, greenness or biomass, or a decrease in early senescence.

In any of the embodiments described herein, the plant may exhibit the alteration of at least one agronomic characteristic when compared, under water limiting conditions, to a control plant not comprising said recombinant DNA construct (or said suppression DNA construct).

In any of the embodiments described herein, the plant may exhibit less yield loss relative to the control plants, for example, at least 25%, at least 20%, at least 15%, at least 10% or at least 5% less yield loss, under water limiting conditions, or would have increased yield, for example, at least 5%, at least 10%, at least 15%, at least 20% or at least 25% increased yield, relative to the control plants under water non-limiting conditions.

“Drought” refers to a decrease in water availability to a plant that, especially when prolonged, can cause damage to the plant or prevent its successful growth (e.g., limiting plant growth or seed yield). “Water limiting conditions” refers to a plant growth environment where the amount of water is not sufficient to sustain optimal plant growth and development. The terms “drought” and “water limiting conditions” are used interchangeably herein.

“Drought tolerance” is a trait of a plant to survive under drought conditions over prolonged periods of time without exhibiting substantial physiological or physical deterioration.

“Drought tolerance activity” of a polypeptide indicates that over-expression of the polypeptide in a transgenic plant confers increased drought tolerance to the transgenic plant relative to a reference or control plant.

“Increased drought tolerance” of a plant is measured relative to a reference or control plant, and is a trait of the plant to survive under drought conditions over prolonged periods of time, without exhibiting the same degree of physiological or physical deterioration relative to the reference or control plant grown under similar drought conditions. Typically, when a transgenic plant comprising a recombinant DNA construct or suppression DNA construct in its genome exhibits increased drought tolerance relative to a reference or control plant, the reference or control plant does not comprise in its genome the recombinant DNA construct or suppression DNA construct.

“Triple stress” as used herein refers to the abiotic stress exerted on the plant by the combination of drought stress, high temperature stress and high light stress.

The terms “heat stress” and “temperature stress” are used interchangeably herein, and are defined as where ambient temperatures are hot enough for sufficient time that they cause damage to plant function or development, which might be reversible or irreversible in damage.“High temperature” can be either “high air temperature” or “high soil temperature”, “high day temperature” or “high night temperature, or a combination of more than one of these.

In one embodiment of the disclosure, the ambient temperature can be in the range of 30° C. to 36° C. In one embodiment of the disclosure, the duration for the high temperature stress could be in the range of 1-16 hours.

“High light intensity” and “high irradiance” and “light stress” are used interchangeably herein, and refer to the stress exerted by subjecting plants to light intensities that are high enough for sufficient time that they cause photoinhibition damage to the plant.

In one embodiment of the disclosure, the light intensity can be in the range of 250 μE to 450 μE. In one embodiment of the disclosure, the duration for the high light inetnsity stress could be in the range of 12-16 hours.

“Triple stress tolerance” is a trait of a plant to survive under the combined stress conditions of drought, high temperature and high light intensity over prolonged periods of time without exhibiting substantial physiological or physical deterioration.

“Paraquat” is an herbicide that exerts oxidative stress on the plants. Paraquat, a bipyridylium herbicide, acts by intercepting electrons from the electron transport chain at PSI. This reaction results in the production of bipyridyl radicals that readily react with dioxygen thereby producing superoxide. Paraquat tolerance in a plant has been associated with the scavenging capacity for oxyradicals (Lannelli, M. A. et al (1999) J Exp Botany, Vol. 50, No. 333, pp. 523-532). Paraquat resistant plants have been reported to have higher tolerance to other oxidative stresses as well.

“Paraquat stress” is defined as stress exerted on the plants by subjecting them to Paraquat concentrations ranging from 0.03 to 0.3 μM.

Many adverse environmental conditions such as drought, salt stress, and use of herbicide promote the overproduction of reactive oxygen species (ROS) in plant cells. ROS such as singlet oxygen, superoxide radicals, hydrogen peroxide (H₂O₂), and hydroxyl radicals are believed to be the major factor responsible for rapid cellular damage due to their high reactivity with membrane lipids, proteins, and DNA (Mittler, R. (2002) Trends Plant Sci Vol. 7 No. 9).

A polypeptide with “triple stress tolerance activity” indicates that over-expression of the polypeptide in a transgenic plant confers increased triple stress tolerance to the transgenic plant relative to a reference or control plant. A polypeptide with “paraquat stress tolerance activity” indicates that over-expression of the polypeptide in a transgenic plant confers increased Paraquat stress tolerance to the transgenic plant relative to a reference or control plant.

Typically, when a transgenic plant comprising a recombinant DNA construct or suppression DNA construct in its genome exhibits increased stress tolerance relative to a reference or control plant, the reference or control plant does not comprise in its genome the recombinant DNA construct or suppression DNA construct.

One of ordinary skill in the art is familiar with protocols for simulating drought conditions and for evaluating drought tolerance of plants that have been subjected to simulated or naturally-occurring drought conditions. For example, one can simulate drought conditions by giving plants less water than normally required or no water over a period of time, and one can evaluate drought tolerance by looking for differences in physiological and/or physical condition, including (but not limited to) vigor, growth, size, or root length, or in particular, leaf color or leaf area size. Other techniques for evaluating drought tolerance include measuring chlorophyll fluorescence, photosynthetic rates and gas exchange rates.

A drought stress experiment may involve a chronic stress (i.e., slow dry down) and/or may involve two acute stresses (i.e., abrupt removal of water) separated by a day or two of recovery. Chronic stress may last 8-10 days. Acute stress may last 3-5 days. The following variables may be measured during drought stress and well watered treatments of transgenic plants and relevant control plants:

The variable “% area chg_start chronic-acute2” is a measure of the percent change in total area determined by remote visible spectrum imaging between the first day of chronic stress and the day of the second acute stress.

The variable “% area chg_start chronic-end chronic” is a measure of the percent change in total area determined by remote visible spectrum imaging between the first day of chronic stress and the last day of chronic stress.

The variable “% area chg_start chronic-harvest” is a measure of the percent change in total area determined by remote visible spectrum imaging between the first day of chronic stress and the day of harvest.

The variable “% area chg_start chronic-recovery24 hr” is a measure of the percent change in total area determined by remote visible spectrum imaging between the first day of chronic stress and 24 hrs into the recovery (24 hrs after acute stress 2).

The variable “psii_acute1” is a measure of Photosystem II (PSII) efficiency at the end of the first acute stress period. It provides an estimate of the efficiency at which light is absorbed by PSII antennae and is directly related to carbon dioxide assimilation within the leaf.

The variable “psii_acute2” is a measure of Photosystem II (PSII) efficiency at the end of the second acute stress period. It provides an estimate of the efficiency at which light is absorbed by PSII antennae and is directly related to carbon dioxide assimilation within the leaf.

The variable “fv/fm_acute1” is a measure of the optimum quantum yield (Fv/Fm) at the end of the first acute stress-(variable fluorescence difference between the maximum and minimum fluorescence/maximum fluorescence)

The variable “fv/fm_acute2” is a measure of the optimum quantum yield (Fv/Fm) at the end of the second acute stress-(variable flourescence difference between the maximum and minimum fluorescence/maximum fluorescence).

The variable “leaf rolling_harvest” is a measure of the ratio of top image to side image on the day of harvest.

The variable “leaf rolling_recovery24 hr” is a measure of the ratio of top image to side image 24 hours into the recovery.

The variable “Specific Growth Rate (SGR)” represents the change in total plant surface area (as measured by Lemna Tec Instrument) over a single day (Y(t)=Y0*e^{r*t}). Y(t)=Y0*e^{r*t} is equivalent to % change in Y/Δ t where the individual terms are as follows: Y(t)=Total surface area at t; Y0=Initial total surface area (estimated); r=Specific Growth Rate day^{-1,} and t=Days After Planting (“DAP”).

The variable “shoot dry weight” is a measure of the shoot weight 96 hours after being placed into a 104° C. oven.

The variable “shoot fresh weight” is a measure of the shoot weight immediately after being cut from the plant.

The Examples below describe some representative protocols and techniques for simulating drought conditions and/or evaluating drought tolerance.

One can also evaluate drought tolerance by the ability of a plant to maintain sufficient yield (at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% yield) in field testing under simulated or naturally-occurring drought conditions (e.g., by measuring for substantially equivalent yield under drought conditions compared to non-drought conditions, or by measuring for less yield loss under drought conditions compared to a control or reference plant).

One of ordinary skill in the art would readily recognize a suitable control or reference plant to be utilized when assessing or measuring an agronomic characteristic or phenotype of a transgenic plant in any embodiment of the present disclosure in which a control plant is utilized (e.g., compositions or methods as described herein). For example, by way of non-limiting illustrations:

1. Progeny of a transformed plant which is hemizygous with respect to a recombinant DNA construct (or suppression DNA construct), such that the progeny are segregating into plants either comprising or not comprising the recombinant DNA construct (or suppression DNA construct): the progeny comprising the recombinant DNA construct (or suppression DNA construct) would be typically measured relative to the progeny not comprising the recombinant DNA construct (or suppression DNA construct) (i.e., the progeny not comprising the recombinant DNA construct (or the suppression DNA construct) is the control or reference plant).

2. Introgression of a recombinant DNA construct (or suppression DNA construct) into an inbred line, such as in maize, or into a variety, such as in soybean: the introgressed line would typically be measured relative to the parent inbred or variety line (i.e., the parent inbred or variety line is the control or reference plant).

3. Two hybrid lines, where the first hybrid line is produced from two parent inbred lines, and the second hybrid line is produced from the same two parent inbred lines except that one of the parent inbred lines contains a recombinant DNA construct (or suppression DNA construct): the second hybrid line would typically be measured relative to the first hybrid line (i.e., the first hybrid line is the control or reference plant).

4. A plant comprising a recombinant DNA construct (or suppression DNA construct): the plant may be assessed or measured relative to a control plant not comprising the recombinant DNA construct (or suppression DNA construct) but otherwise having a comparable genetic background to the plant (e.g., sharing at least 90%, 91%, 92%, 93%, 94%, 95%, 96%, 97%, 98%, 99%, or 100% sequence identity of nuclear genetic material compared to the plant comprising the recombinant DNA construct (or suppression DNA construct)). There are many laboratory-based techniques available for the analysis, comparison and characterization of plant genetic backgrounds; among these are Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLP®s), and Simple Sequence Repeats (SSRs) which are also referred to as Microsatellites.

Furthermore, one of ordinary skill in the art would readily recognize that a suitable control or reference plant to be utilized when assessing or measuring an agronomic characteristic or phenotype of a transgenic plant would not include a plant that had been previously selected, via mutagenesis or transformation, for the desired agronomic characteristic or phenotype.

Methods:

Methods include but are not limited to methods for increasing drought tolerance in a plant, methods for evaluating drought tolerance in a plant, methods for altering an agronomic characteristic in a plant, methods for determining an alteration of an agronomic characteristic in a plant, and methods for producing seed. The plant may be a monocotyledonous or dicotyledonous plant, for example, a maize or soybean plant. The plant may also be sunflower, sorghum, canola, wheat, alfalfa, cotton, rice, barley, millet, sugar cane or sorghum. The seed may be a maize or soybean seed, for example, a maize hybrid seed or maize inbred seed.

Methods include but are not limited to the following:

A method for transforming a cell (or microorganism) comprising transforming a cell (or microorganism) with any of the isolated polynucleotides or recombinant DNA constructs of the present disclosure. The cell (or microorganism) transformed by this method is also included. In particular embodiments, the cell is eukaryotic cell, e.g., a yeast, insect or plant cell, or prokaryotic, e.g., a bacterial cell. The microorganism may be Agrobacterium, e.g. Agrobacterium tumefaciens or Agrobacterium rhizogenes.

A method for producing a transgenic plant comprising transforming a plant cell with any of the isolated polynucleotides or recombinant DNA constructs (including suppression DNA constructs) of the present disclosure and regenerating a transgenic plant from the transformed plant cell. The disclosure is also directed to the transgenic plant produced by this method, and transgenic seed obtained from this transgenic plant. The transgenic plant obtained by this method may be used in other methods of the present disclosure.

A method of altering the level of expression of a polypeptide of the disclosure in a host cell comprising: (a) transforming a host cell with a recombinant DNA construct of the present disclosure; and (b) growing the transformed host cell under conditions that are suitable for expression of the recombinant DNA construct wherein expression of the recombinant DNA construct results in production of altered levels of the polypeptide of the disclosure in the transformed host cell.

A method of increasing drought tolerance in a plant, comprising: (a) introducing into a regenerable plant cell a suppression DNA constructcomprising at least one regulatory sequence (for example, a promoter functional in a plant) operably linked to all or part of (i) a nucleic acid sequence encoding a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:47, 48, 50 or 51, or (ii) a full complement of the nucleic acid sequence of (a)(i); and (b) regenerating a transgenic plant from the regenerable plant cell after step (a), wherein the transgenic plant comprises in its genome the suppression DNA construct and exhibits increased drought tolerance when compared to a control plant not comprising the suppression DNA construct. The method may further comprise (c) obtaining a progeny plant derived from the transgenic plant, wherein said progeny plant comprises in its genome the suppression DNA construct and exhibits increased drought tolerance when compared to a control plant not comprising the suppression DNA construct.

A method of increasing drought tolerance in a plant, comprising: (a) introducing into a regenerable plant cell a suppression DNA construct comprising at least one regulatory sequence (for example, a promoter functional in a plant) operably linked to a region derived from all or part of a sense strand or antisense strand of a target gene of interest, said region having a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to said all or part of a sense strand or antisense strand from which said region is derived, and wherein said target gene of interest encodes a Slm1 polypeptide; and (b) regenerating a transgenic plant from the regenerable plant cell after step (a), wherein the transgenic plant comprises in its genome the suppression DNA construct and exhibits increased drought tolerance when compared to a control plant not comprising the suppression DNA construct. The method may further comprise (c) obtaining a progeny plant derived from the transgenic plant, wherein said progeny plant comprises in its genome the suppression DNA construct and exhibits increased drought tolerance when compared to a control plant not comprising the suppression DNA construct.

A method of selecting for (or identifying) drought tolerance in a plant, comprising (a) obtaining a transgenic plant, wherein the transgenic plant comprises in its genome a suppression DNA construct comprising at least one regulatory sequence (for example, a promoter functional in a plant) operably linked to all or part of (i) a nucleic acid sequence encoding a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:47, 48, 50 or 51, or (ii) a full complement of the nucleic acid sequence of (a)(i); (b) obtaining a progeny plant derived from said transgenic plant, wherein the progeny plant comprises in its genome the suppression DNA construct; and (c) selecting for (or identifying) the progeny plant that exhibits drought tolerance compared to a control plant not comprising the suppression DNA construct.

A method of selecting for (or identifying) drought tolerance in a plant, comprising (a) obtaining a transgenic plant, wherein the transgenic plant comprises in its genome a suppression DNA construct comprising at least one regulatory sequence (for example, a promoter functional in a plant) operably linked to a region derived from all or part of a sense strand or antisense strand of a target gene of interest, said region having a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to said all or part of a sense strand or antisense strand from which said region is derived, and wherein said target gene of interest encodes a Slm1 polypeptide; (b) obtaining a progeny plant derived from the transgenic plant, wherein the progeny plant comprises in its genome the suppression DNA construct; and (c) selecting for (or identifying) the progeny plant that exhibits drought tolerance compared to a control plant not comprising the suppression DNA construct.

A method of selecting for (or identifying) an alteration of an agronomic characteristic in a plant, comprising (a) obtaining a transgenic plant, wherein the transgenic plant comprises in its genome a suppression DNA construct comprising at least one regulatory sequence (for example, a promoter functional in a plant) operably linked to all or part of (i) a nucleic acid sequence encoding a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:47, 48, 50 or 51, or (ii) a full complement of the nucleic acid sequence of (i); (b) obtaining a progeny plant derived from said transgenic plant, wherein the progeny plant comprises in its genome the suppression DNA construct; and (c) selecting (or identifying) the progeny plant that exhibits an alteration in at least one agronomic characteristic when compared, optionally under water limiting conditions, to a control plant not comprising the suppression DNA construct.

A method of selecting for (or identifying) an alteration of an agronomic characteristic in a plant, comprising (a) obtaining a transgenic plant, wherein the transgenic plant comprises in its genome a suppression DNA construct comprising at least one regulatory sequence (for example, a promoter functional in a plant) operably linked to a region derived from all or part of a sense strand or antisense strand of a target gene of interest, said region having a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to said all or part of a sense strand or antisense strand from which said region is derived, and wherein said target gene of interest encodes a Slm1 polypeptide; (b) obtaining a progeny plant derived from said transgenic plant, wherein the progeny plant comprises in its genome the suppression DNA construct; and (c) selecting (or identifying) the progeny plant that exhibits an alteration in at least one agronomic characteristic when compared, optionally under water limiting conditions, to a control plant not comprising the suppression DNA construct.

A method of selecting for (or identifying) a maize plant with a decrease in early senescence, comprising (a) obtaining a transgenic maize plant displaying early senescence to a non-transgenic maize plant; (b) obtaining a second transgenic maize plant containing the transgenes of the maize plant of step (a) and additionally a mutant slm1 allele; and (c) selecting (or identifying) the second transgenic maize plant of step (b) that displays a decrease in early senescence. The transgenic plant of step (a) may comprise a cry34 and a cry35 transgene. The mutant slm1 allele of step (b) may comprise SEQ ID NO:73. A maize plant or a maize seed produced by the above method.

A method of selecting for (or identifying) a maize plant with an increase in drought tolerance, comprising (a) obtaining a transgenic maize plant; (b) obtaining a second transgenic maize plant containing the transgenes of the maize plant of step (a) and additionally a mutant slm1 allele; and (c) selecting (or identifying) the second transgenic maize plant of step (b) that displays an increase in drought tolerance. The transgenic plant of step (a) may comprise a cry34 and a cry35 transgene. The mutant slm1 allele of step (b) may comprise SEQ ID NO:73. A maize plant or a maize seed produced by the above method.

A method of selecting for (or identifying) a maize plant with an increase in paraquat resistance, comprising (a) obtaining a transgenic maize plant; (b) obtaining a second transgenic maize plant containing the transgene of the maize plant of step (a) and additionally a mutant slm1 allele; and (c) selecting (or identifying) the second transgenic maize plant of step (b) that displays an increase in paraquat resistance. The mutant slm1 allele of step (b) may comprise SEQ ID NO:73. A maize plant or a maize seed produced by the above method.

A method of selecting for (or identifying) a maize plant with an increase in triple stress resistance, comprising (a) obtaining a transgenic maize plant; (b) obtaining a second transgenic maize plant containing the transgene of the maize plant of step (a) and additionally a mutant slm1 allele; and (c) selecting (or identifying) the second transgenic maize plant of step (b) that displays an increase in triple stress resistance. The mutant slm1 allele of step (b) may comprise SEQ ID NO:73. A maize plant or a maize seed produced by the above method.

A method of producing seed (for example, seed that can be sold as a drought tolerant product offering) comprising any of the preceding methods, and further comprising obtaining seeds from said progeny plant, wherein said seeds comprise in their genome said recombinant DNA construct (or suppression DNA construct).

In any of the preceding methods or any other embodiments of methods of the present disclosure, in said introducing step said regenerable plant cell may comprise a callus cell, an embryogenic callus cell, a gametic cell, a meristematic cell, or a cell of an immature embryo. The regenerable plant cells may derive from an inbred maize plant.

In any of the preceding methods or any other embodiments of methods of the present disclosure, said regenerating step may comprise the following: (i) culturing said transformed plant cells in a media comprising an embryogenic promoting hormone until callus organization is observed; (ii) transferring said transformed plant cells of step (i) to a first media which includes a tissue organization promoting hormone; and (iii) subculturing said transformed plant cells after step (ii) onto a second media, to allow for shoot elongation, root development or both.

In any of the preceding methods or any other embodiments of methods of the present disclosure, the at least one agronomic characteristic may be selected from the group consisting of: abiotic stress tolerance, early senescence, greenness, yield, growth rate, biomass, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, amino acid content in a vegetative tissue, total plant protein content, fruit protein content, seed protein content, protein content in a vegetative tissue, drought tolerance, nitrogen uptake, root lodging, harvest index, stalk lodging, plant height, ear height, ear length, salt tolerance, early seedling vigor and seedling emergence under low temperature stress. The alteration of at least one agronomic characteristic may be an increase in yield, greenness or biomass, or a decrease in early senescence.

In any of the preceding methods or any other embodiments of methods of the present disclosure, the plant may exhibit the alteration of at least one agronomic characteristic when compared, under water limiting conditions, to a control plant not comprising said recombinant DNA construct (or said suppression DNA construct).

In any of the preceding methods or any other embodiments of methods of the present disclosure, alternatives exist for introducing into a regenerable plant cell a recombinant DNA construct comprising a polynucleotide operably linked to at least one regulatory sequence. For example, one may introduce into a regenerable plant cell a regulatory sequence (such as one or more enhancers, optionally as part of a transposable element), and then screen for an event in which the regulatory sequence is operably linked to an endogenous gene encoding a polypeptide of the instant disclosure.

The introduction of recombinant DNA constructs of the present disclosure into plants may be carried out by any suitable technique, including but not limited to direct DNA uptake, chemical treatment, electroporation, microinjection, cell fusion, infection, vector-mediated DNA transfer, bombardment, or Agrobacterium-mediated transformation. Techniques for plant transformation and regeneration have been described in International Patent Publication WO 2009/006276, the contents of which are herein incorporated by reference.

The development or regeneration of plants containing the foreign, exogenous isolated nucleic acid fragment that encodes a protein of interest is well known in the art. The regenerated plants may be self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the present disclosure containing a desired polypeptide is cultivated using methods well known to one skilled in the art.

### EXAMPLES

The present disclosure is further illustrated in the following Examples, in which parts and percentages are by weight and degrees are Celsius, unless otherwise stated. It should be understood that these Examples, while indicating embodiments of the disclosure, are given by way of illustration only. From the above discussion and these Examples, one skilled in the art can ascertain the essential characteristics of this disclosure, and without departing from the spirit and scope thereof, can make various changes and modifications of the disclosure to adapt it to various usages and conditions. Thus, various modifications of the disclosure in addition to those shown and described herein will be apparent to those skilled in the art from the foregoing description. Such modifications are also intended to fall within the scope of the appended claims.

### Example 1

### Populations Used to Fine Map the Slm1 Locus

To fine map and isolate the slm1 locus, we developed an isogenic line heterozygous for slm1 locus from the initial cross between Mo20W and les23:Va35 after seven to eight backcrosses with the recurrent parent les23:Va35. We selfed these BC7F1 and BC8F1 to generate two F2 populations, and backcrossed the BC8F1 to les23:Va35 to establish a backcross population. We phenotyped and genotyped these segregating populations with PCR based markers, which located slm1 between c0176e15_{—}42b and c0176e15_{—}7_{—}8.

### Example 2

### Fine Mapping of the Slm1 Locus

For Slm1, we used 8 plates of leaf tissue from a BC₇F₂ mapping population to extract DNA for fine mapping. We used a total of 567 individuals. Two of the plates contained samples of wild-type individuals (no lesion formation), two plates were mutant individuals (severe lesion formation), and four of the plates contained heterozygous individuals (intermediate lesion formation). Instead of using SSR markers, Insertion-Deletion Polymorphism (IDP) markers roughly corresponding to the previously identified flanking markers of bnlg1045 and bnlg1316 were tested and used to delimitate the outer boundaries of the Slm1 interval. IDP3952 (174.2 cM) was found to be the left flanking marker, corresponding to bnlg1045, and IDP8680 (197.8) was the right flanking marker corresponding to bnlg1316

Primers IDP3952_For (SEQ ID NO: 1) and IDP3952_Rev (SEQ ID NO: 2) were used to amplify the IDP3952 locus. The PCR product was run on a gel and the banding pattern was analyzed to determine the genotypes at this locus.

Primers IDP8680_For (SEQ ID NO: 3) and IDP8680_Rev (SEQ ID NO: 4) were used to amplify the IDP8680 locus. The PCR product was run on a gel and the banding pattern was analyzed to determine the genotypes at this locus.

Following the identification of the flanking markers IDP3952 and IDP8680, additional markers were developed to further narrow the Slm1 interval. MZA primer sequences were used to amplify and sequence the corresponding region in Va35 and Mo20W. Sequencing results were analyzed for the presence of a polymorphism which added or removed a restriction enzyme site. MZA3156 (187.7 cM) and MZA5988 (189.4 cM) were used to narrow the Slm1 interval to just over 1 BAC in length.

Primers MZA3156_For (SEQ ID NO: 5) and MZA3156_Rev (SEQ ID NO: 6) were used to amplify the MZA3156 locus. The PCR was digested with Ddel and the banding pattern was analyzed to determine the genotypes at this locus.

Primers MZA5988_For (SEQ ID NO: 7) and MZA5988_Rev (SEQ ID NO: 8) were used to amplify the MZA5988 locus. PCR product for this reaction was used as template for a second reaction using the primers MZA5988_ForNest (SEQ ID NO: 9) and MZA5988_RevNest (SEQ ID NO: 10). This PCR product was digested with BsiHKAl and the banding pattern was analyzed to determine the genotypes at this locus.

Within this ˜1 BAC interval, there were a relatively small number of genes. One gene, annotated as a putative R gene, was deemed a likely candidate for slm1.

### Example 3

### Sequence Analysis of the Slm1 Locus

R genes are involved in the hypersensitive response, which results in the formation of lesions and is reminiscent of the lesion phenotype seen in this population. Therefore, we decided to sequence the putative R gene in Va35 and Mo20W to look for any obvious difference. Nine sets of nested primers (SEQ ID NO: 11-46) were designed spanning the entire gene for sequencing. These primers were designed based on the available B73 sequence. Sequencing results showed that in Va35, the putative R gene is a complete and intact gene (Genomic Sequence: SEQ ID NO: 47; CDS: SEQ ID NO: 48; Protein Sequence: SEQ ID NO: 49) while in Mo20W, among other differences, there is a 2 bp insertion in the coding sequence which results in a frame shift and an early stop codon in the protein sequence (Genomic Sequence: SEQ ID NO: 50; CDS: SEQ ID NO: 51; Protein Sequence: SEQ ID NO: 52). Therefore, the putative R gene was identified as our main candidate gene.

Primer pairs c0176e15_{—}21 For (SEQ ID NO: 11) and c0176e15_{—}21 Rev (SEQ ID NO: 12), c0176e15_{—}22 For (SEQ ID NO: 15) and c0176e15_{—}22 Rev (SEQ ID NO: 16), c0176e15_{—}23 For (SEQ ID NO: 19) and c0176e15_{—}23 Rev (SEQ ID NO: 20), c0176e15_{—}24 For (SEQ ID NO: 23) and c0176e15_{—}24 Rev (SEQ ID NO: 24), c0176e15_{—}25 For (SEQ ID NO: 27) and c0176e15_{—}25 Rev (SEQ ID NO: 28), c0176e15_{—}26 For (SEQ ID NO: 31) and c0176e15_{—}26 Rev (SEQ ID NO: 32), c0176e15_{—}27 For (SEQ ID NO: 35) and c0176e15_{—}27 Rev (SEQ ID NO: 36), c0176e15_{—}28 For (SEQ ID NO: 39) and c0176e15_{—}28 Rev (SEQ ID NO: 40), and c0176e15_{—}29 For (SEQ ID NO: 43) and c0176e15_{—}29 Rev (SEQ ID NO: 44) were used to amplify the genomic region spanning the putative R gene. PCR products from these reactions were used as templates for second reactions using the corresponding primer pairs: c0176e15_{—}21 ForNest (SEQ ID NO: 13) and c0176e15_{—}21 RevNest (SEQ ID NO: 14), c0176e15_{—}22 ForNest (SEQ ID NO: 17) and c0176e15_{—}22 RevNest (SEQ ID NO: 18), c0176e15_{—}23 ForNest (SEQ ID NO: 21) and c0176e15_{—}23 RevNest (SEQ ID NO: 22), c0176e15_{—}24 ForNest (SEQ ID NO: 25) and c0176e15_{—}24 RevNest (SEQ ID NO: 26), c0176e15_{—}25 ForNest (SEQ ID NO: 29) and c0176e15_{—}25 RevNest (SEQ ID NO: 30), c0176e15_{—}26 ForNest (SEQ ID NO: 33) and c0176e15_{—}26 RevNest (SEQ ID NO: 34), c0176e15_{—}27 ForNest (SEQ ID NO: 37) and c0176e15_{—}27 RevNest (SEQ ID NO: 38), c0176e15_{—}28 ForNest (SEQ ID NO: 41) and c0176e15_{—}28 RevNest (SEQ ID NO: 42), and c0176e15_{—}29 ForNest (SEQ ID NO: 45) and c0176e15_{—}29 RevNest (SEQ ID NO: 46).

For further support that the putative R gene is the causative gene for Slm1, additional mapping was done within the ˜1 BAC interval. PCR primers were designed from low copy regions based on available B73 sequence. If a size difference was observed in the products from Va35 and Mo20W, the marker was used as an IDP. If there was no easily discernible size difference, PCR products were sequenced and analyzed for the presence of a polymorphism which caused a change in a restriction enzyme site. Using the markers, c0176e15_{—}45/46 and c0176e15_{—}8/7, the Slm1 interval was narrowed down to a ˜12kb region in which the putative R gene was the only gene present.

Primer c0176e15_{—}45 For (SEQ ID NO: 53) and c0176e15_{—}46 Rev (SEQ ID NO: 54) were used to amplify a region on the BAC, c0176e15. PCR product for this reaction was used as a template for a second reaction using the primers c0176e15_{—}45 ForNest (SEQ ID NO: 55) and c0176e15_{—}46 RevNest (SEQ ID NO: 56). This PCR product was digested with BsaJl and the banding pattern was analyzed to determine the genotypes at this locus.

Primers c0176e15_{—}8 For (SEQ ID NO: 57) and c0176e15_{—}7 RevNest (SEQ ID NO: 58) were used to amplify a region on the BAC, c0176e15. The PCR product was run on a gel and the difference in band sizes were analyzed to determine the genotypes at this locus.

### Example 4

### Validation of the Slm1 Locus

The putative R gene candidate was validated by the use of independent EMS and Mu-insertion alleles.

To confirm the identity of slm1, we searched UniFormMu stock lines and found one stock with Mu insertion at 2289 by from the start codon of slm1(named as Slm1-Mu*-W22). We then crossed this line with les23:Va35 and established a F2 population, in which expression of les23 phenotype co-segregates with the genotype in slm1 locus based on the analysis of 94 F2 individuals. In addition, sequence analyses of two independent slm1 alleles recovered from about 15,000 progeny in a direct EMS mutagenesis experiment reveal missense point mutations: i.e., G2018A (Gly673Glu) in the mutant Slm1-EMS8 and A2180G (Asp727Gly) in the mutant Slm1-EMS15. Our data suggest that slm1 encodes a typical NBS-LRR R gene and a functional R gene is required for the cell death phenotype underlying the les23 mutation.

### Example 5

### Fine Mapping of the Les23 Locus

We used ˜575 seed from an F2 population of B73 crossed to les23 in the Va35 background. These plants were grown and phenotyped in a greenhouse at the DuPont Experimental Station in Wilmington, Del. Many markers were developed that allowed us to narrow the les23 interval to ˜10 cM. A lack of recombinants prevented further progress. Although not the closest flanking markers, IDP200 (119.5 cM) and MZA6815 (132.7 cM) were used to screen additional individuals as they were the clearest and easiest to use markers.

Primers MZA6815_For (SEQ ID NO: 59) and MZA6815_Rev (SEQ ID NO: 60) were used to amplify the MZA6815 locus. The PCR product was run on a gel and the difference in band sizes were analyzed to determine the genotypes at this locus.

Primers IDP200_For (SEQ ID NO: 61) and IDP200_Rev (SEQ ID NO: 62) were used to amplify the IDP200 locus. The PCR product was run on a gel and the difference in band sizes were analyzed to determine the genotypes at this locus.

For further les23 mapping, we used an additional ˜1500 seed from the B73 crossed to les23 in the Va35 background mapping population. These plants were also grown in a greenhouse at DuPont Experimental Station in Wilmington, Del. The plants were sampled and genotyped with the IDP markers, MZA6815 and IDP200. Recombinants were phenotyped and used for additional mapping. Delimitating a large physical interval, MZA760 (127.2 cM) and MZA15537 (127.7) were the closest flanking markers that were able to be designed due to the proximity of les23 to the centromere.

Primers MZA760_For (SEQ ID NO: 63) and MZA760_Rev (SEQ ID NO: 64) were used to amplify the MZA760 locus. The PCR was digested with BsrDl and the banding pattern was analyzed to determine the genotypes at this locus.

Primers MZA15537_For (SEQ ID NO: 65) and MZA15537_Rev (SEQ ID NO: 66) were used to amplify the MZA15537 locus. The PCR was digested with BsrDl and the banding pattern was analyzed to determine the genotypes at this locus.

Instead of attempting to continue to narrow the les23 interval, we decided to examine the list of genes in the region and determine if there were any obvious candidates, given that the causative gene for Slm1 was a putative R gene.

### Example 6

### Sequence Analysis and Validation of the Les23 Locus

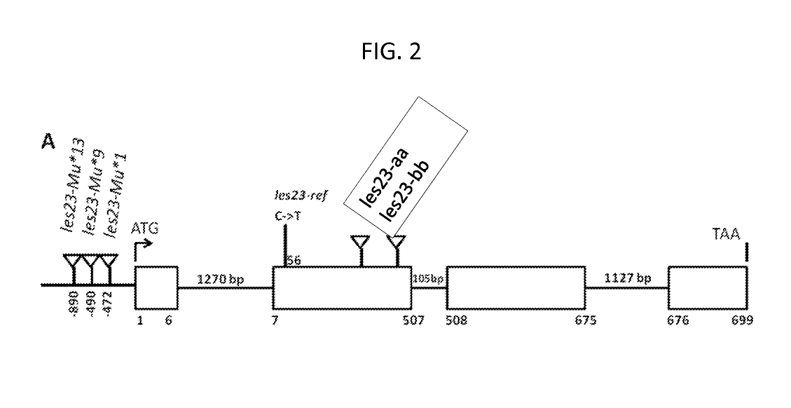

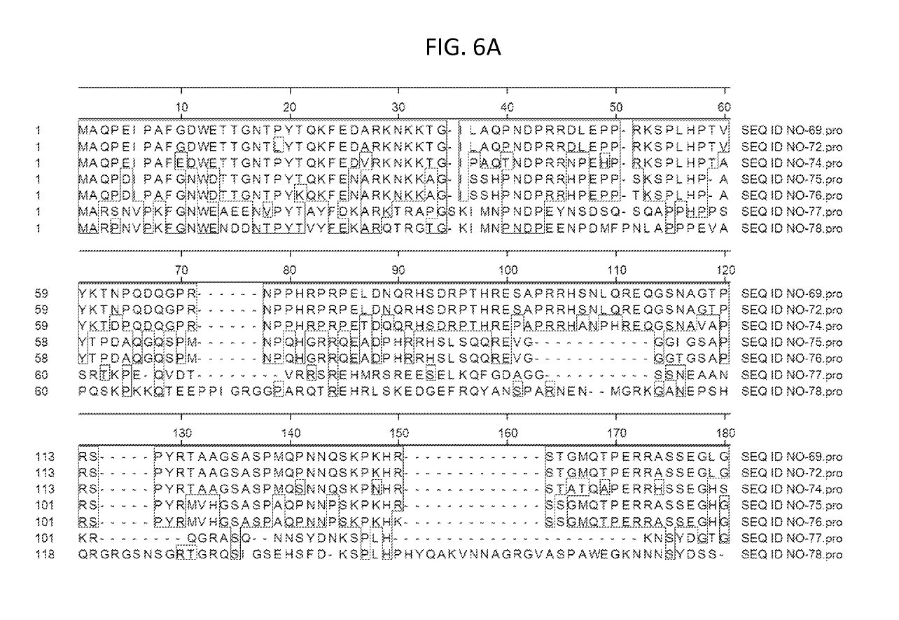

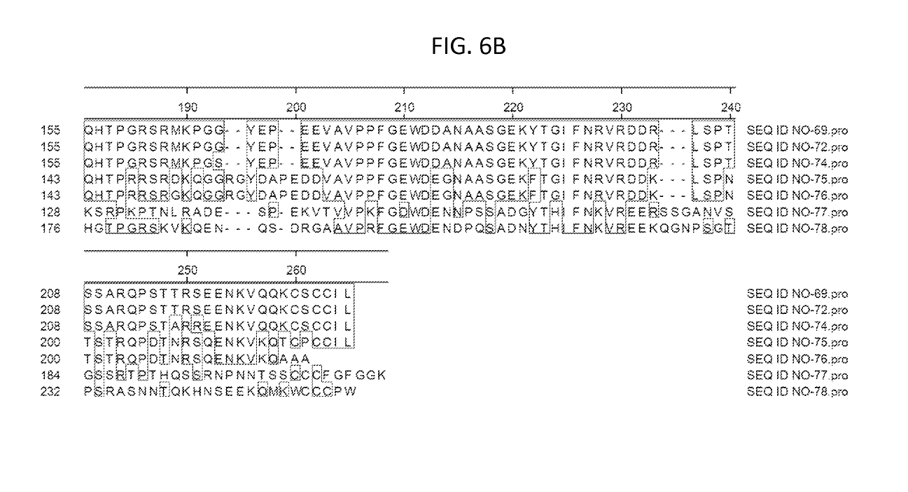

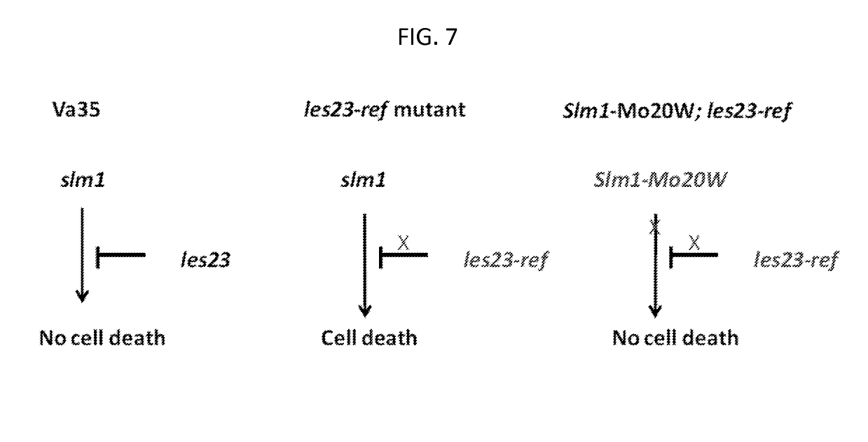

Fine mapping localized les23 to an interval with about 64 putative genes, and one of them was annotated as RPM1 interacting (RIN). In Arabidopsis, RIN4 interacts genetically and physically with RPM1 and RPS2, which belong to CC-NBS-LRR R class proteins. Since Slm1 encodes the same class of R protein, we hypothesized that LES23 may be a maize homolog of AtRIN4. To test this idea, we first cloned RIN4 CDS homolog from maize les23 mutant and WT, and we found that a missense point mutation in rin4 from les23:Va35 (C55T) resulted in alteration of one amino acid (P19L) that is conserved among rin4 homologues from eudicots and monocots (FIG. 6A-6B). Further molecular characterization of independent alleles form Mu tagging experiments revealed differential insertions of Mu elements in the promoter region of rin4 in three mutants (all within the range of 1 Kb from the translation start codon) and in the exon 2 of rin4 in two other mutants (FIG. 2). All five mutants show the same lesion phenotype as les23:Va35. Our data strongly support that les23 encodes the maize homolog of Atrin4 and normal functional les23 gene negatively regulates cell death process.

The relevant sequences are the following: Wild Type Genomic Sequence: SEQ ID NO: 67; Wild Type CDS: SEQ ID NO: 68; Wild Type Protein Sequence: SEQ ID NO: 69; les23-ref Mutant Genomic Sequence: SEQ ID NO: 70; les23-ref Mutant CDS: SEQ ID NO: 71; les23-ref Mutant Protein Sequence: SEQ ID NO: 72. The les23-ref was an EMS mutagenized mutant in the background of opaque:Va35 and propagated by repeated sib mating between homozygous mutants and heterozygous wild-type plants (Penning et al. 2004 Genome 47:961-969)

### Example 7

### Interactions of SLM1 and LES23 Proteins

Genetic analysis demonstrated that slm1 wt allele is required for the phenotypic expression of les23-ref in the Va35 background. Since Rpm1 protein is known to interact directly with RIN4 proteins, one possibility is that SLM1 directly interacts with and affects the activity of RIN4. To test this possibility a yeast two-hybrid (Y2H) analysis was carried out.

Y2H assays were performed with the GAL4 system according to the instructions from the manufacturer (Stratagene). The full-length and partial (561 bp from the 5′ end) cDNA from Va35, and the truncated cDNA from Mo20W of Slm1 were cloned into pAD-GAL4-2.1 to generate a DNA-binding domain bait protein fusion. The full-length les23 cDNA from Va35 was cloned into pBD-GAL4 to generate an activation domain prey protein fusion. Interactions were tested for complementation of His auxotrophy on selective medium lacking His, Leu, and Trp, and LacZ reporter activity (β-galactosidase assay) together with positive and negative controls by cotransforming appropriate plasmids into the yeast YRG-2 strain.

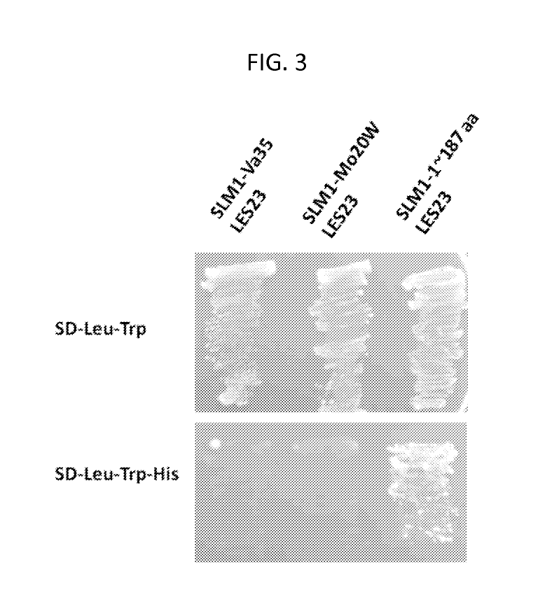

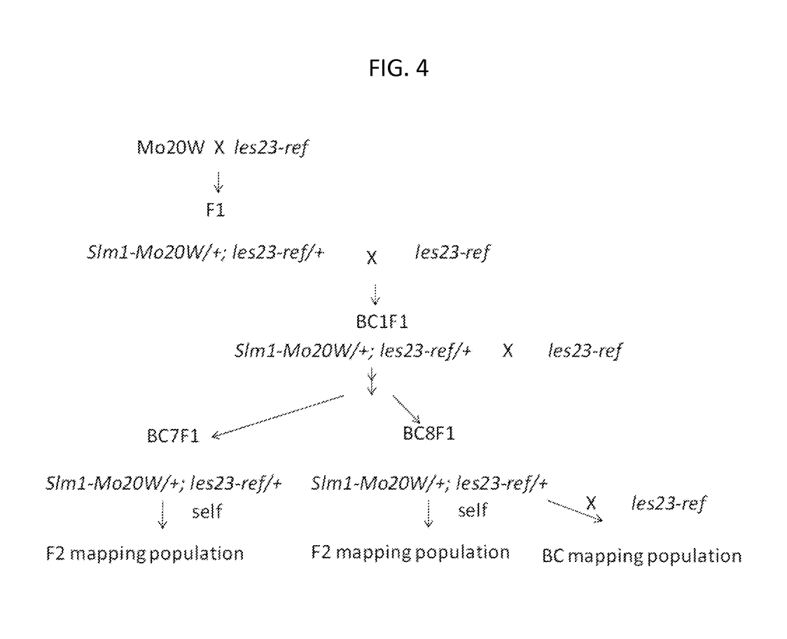

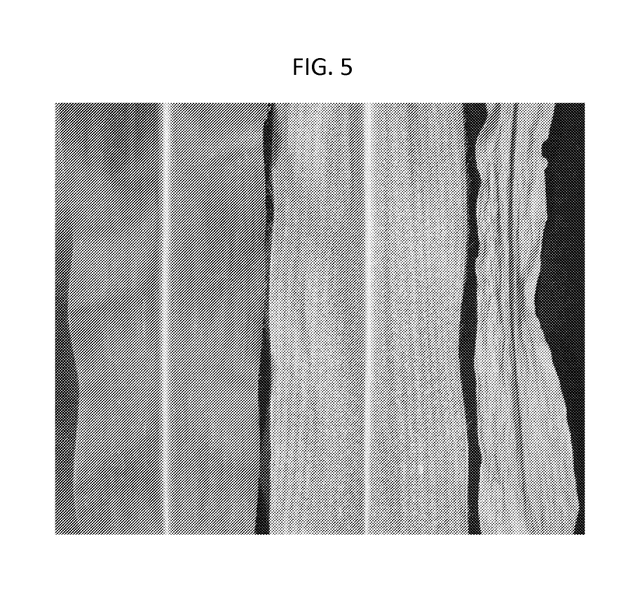

As shown in FIG. 3, we found that full length protein of LES23 physically interacts with N terminus (1-187aa) of SLM1 from Va35, but not with full length protein of SLM1 from Va35 and putative truncated products from Mo20W, which is consistent with previous results from Arabidopsis.

### Example 8

### RPM1/RSP2 and Abiotic Stress Tolerance in Arabidopsis

Assays were performed on Arabidopsis knockout mutants of RPM1 and RSP2 as previously described in PCT International Patent Application No. PCT/US12/62374. No differences were observed between rpm1 and rps2 mutants and wild-type plants in growth rate under normal conditions. In the paraquat assay, rps2 was positive; however, rpm1 has no effect. In the triple stress assay, rpm1 was positive; however, rps2 has no effect. The double mutant of rps2/rpm1 will be generated and tested.

### Example 9

### Slm1 Delays Leaf Fire Associated with HXRW Transgenes

HXRW transgenes (Cry34/35) have been associated with a distinct early senescence (leaf fire) phenotype in certain backgrounds (e.g., HXRW-LF). Data presented in Table 1 suggests that Slm1 can alleviate the leaf fire phenotype.

TABLE 1
Leaf Fire Scores (1-9) in BC3F2 Individuals
from (HXRW-LF) × Mo20W

| | N | Fire2 | Fire3 | Fire4 |
|---|---|---|---|---|
| HXRW-LF slm1 | 154 | 6.32 | 5.44 | 3.95 |
| HXRW-LF WT | 156 | 5.74 | 4.92 | 3.03 |
| Difference | | 0.58 | 0.52 | 0.93 |

### Example 10

### Near-Isogenic Lines (NILs) Tested for Leaf Fire Response

Near-Isogenic Lines (NILs) were created by backcrossing the slm1 donor allele from Mo20W into three recurrent parents containing the HXRW transgene: InbredA-HXRW, InbredB-HXRW, and InbredC-HXRW. The lines were backrossed three generations and then selfed twice, to create BC3S2 NILs both with the Mo20W slm1 allele and without the Mo20W slm1 allele. The InbredA inbred background was previously identified as showing a severe leaf fire phenotype in the presence of the HXRW transgene, and the InbredB and InbredC inbreds showed less severe leaf firing.

The NILs with and without Mo20W were tested for leaf fire response. In the InbredA-HXRW background, 24 NILs with Mo20W slm1 and 24 NILs without Mo20W slm1 were tested. In both the InbredB-HXRW and InbredC-HXRW backgrounds, 15 NILs with Mo20W slm1 and 15 NILs without Mo20W slm1 were tested. The leaf fire phenotype was scored on a 1 to 9 scale, with 1 as most severe leaf firing and 9 as no leaf firing. Means of the NILs were compared, and a Two-sample T test was used to determine statistical significance. The Mo20W slm1 allele caused a 3.1 score increase in the InbredA-HXRW background, which was the genetic background with the most severe response to leaf firing in the presence of the HXRW transgene. In the genetic backgrounds with a less severe firing response, there was a 1.1 score and 0.9 score increase in InbredB-HXRW and InbredC-HXRW, respectively, although neither difference was statistically significant.

TABLE 2

| | InbredA-HXRW | | InbredB-HXRW | | InbredC-HXRW | |
|---|---|---|---|---|---|---|
| | Leaf | SE | Leaf | SE | Leaf | SE |
| | Fire | Mean | Fire | Mean | Fire | Mean |
| NILs with | 5.0 | 0.3 | 7.1 | 0.3 | 5.2 | 0.5 |
| Mo20W slm1 | | | | | | |
| NILs without | 2.0 | 0.3 | 6.0 | 0.8 | 4.3 | 0.5 |
| Mo20W slm1 | | | | | | |
| Difference | 3.1 | | 1.1 | | 0.9 | |
| P-Value | 0.000 | | 0.196 | | 0.194 | |

### Example 11

### Near-Isogenic Lines (NILs) Tested for Yield

The InbredA-HXRW NILs were toperossed to two testers, Tester1 and Tester2, and then were yield tested to determine the effect of the Mo20W slm1 allele in the hybrid. There were 24 two row plots with Mo20W slm1 and 24 two row plots without Mo20W slm1 for each tester. Yield related traits were collected according to standard protocols. The NILs with Mo20W slm1 toperossed to Tester2 had an 11.4 bu/a increase in yield (P-value 0.001), but there was an insignificant increase in the NILs toperossed to Tester1, suggesting a difference in tester effect.

TABLE 3

| | TSTWT | MST | HRVWT | YIELD |
|---|---|---|---|---|
| NILs with Mo20W - Tester1 | 56.6 | 23.1 | 27.4 | 221.0 |
| NILs without Mo20W- | 57.0 | 22.6 | 27.0 | 219.4 |
| Tester1 | | | | |
| Difference | −0.4 | 0.5 | 0.4 | 1.6 |
| P-Value | 0.208 | 0.012 | 0.32 | 0.574 |
| NILs with Mo20W - Tester2 | 54.9 | 24.2 | 29.0 | 230.9 |
| NILs without Mo20W - | 55.3 | 23.8 | 27.4 | 219.5 |
| Tester2 | | | | |
| Difference | −0.5 | 0.3 | 1.6 | 11.4 |
| P-Value | 0.024 | 0.195 | 0.000 | 0.001 |

### Example 12

### Near-Isogenic Lines (NILs) Tested for Drought Tolerance

The NILs with and without Mo20W were tested for response to moderate and severe drought conditions using a hydroponics assay. For the moderate drought condition assay, 14 plants with the Mo20W slm1 allele and 14 plants without the Mo20W slm1 allele were grown under both control conditions and moderate drought conditions. All plants were grown in tubes filled with turface and put in tanks with a timer-controlled circulation of modified Hoaglands nutrient media. The growth rooms were maintained at 26 C/16 Hr (day) and 22 C/8 Hr (night), and the media were maintained at a constant temperature of 65° C. The plants in the control tank (circulate media every two hours) were grown for 38 days, and then the fresh biomass of each plant was measured in grams. The plants in the drought tank were grown for seven days under normal conditions, and then the media was withheld for three days. After the three-day drought period, the plants were returned to normal media conditions for three days. This drought cycle was repeated four times in total. After the final cycle the plants were in normal media for three days, the fresh biomass of each plant was measured in grams.

For the severe drought condition assay, 8 plants with the Mo20W slm1 allele and 8 plants without the Mo20W slm1 allele were grown under both control conditions and severe drought conditions. The plants in the control tank were grown for 33 days, and then the fresh biomass of each plant was measured in grams. The plants in the drought tank were grown for six days under normal conditions, and then the media was withheld for seven days. After the seven-day drought period, the plants were returned to normal media conditions for three days. This drought cycle was repeated three times in total. After the final cycle the plants were in normal media for three days, the fresh biomass of each plant was measured in grams.

The biomass ratio of the plants under drought conditions to plants under control conditions was calculated. Under moderate drought, the InbredA-HXRW plants with the Mo20W-slm1 allele were 41% the size of the control plants, but without the Mo20W-slm1 allele, the drought plants were 21% the size of the control plants.

TABLE 4

| | | Biomass Ratio of Drought | |
|---|---|---|---|
| | | to Control plants | |
| | | Moderate | Severe |
| | | Drought | Drought |
| InbredA-HXRW | without Mo20W-slm1 | 0.21 | 0.14 |
| | with Mo20W-slm1 | 0.41 | 0.18 |
| InbredB-HXRW | without Mo20W-slm1 | 0.22 | 0.16 |
| | with Mo20W-slm1 | 0.21 | 0.20 |
| InbredC-HXRW | without Mo20W-slm1 | 0.32 | 0.19 |
| | with Mo20W-slm1 | 0.25 | 0.19 |

1. (canceled)

2. (canceled)

3. A method of producing a transgenic plant with alteration of an agronomic characteristic, the method comprising:
a. introducing into a regenerable plant cell a recombinant DNA construct comprising an isolated polynucleotide operably linked to at least one regulatory sequence, wherein the polynucleotide encodes a fragment or a variant of a polypeptide having an amino acid sequence of at least 80% sequence identity, based on the Clustal W method of alignment, when compared to SEQ ID NO:49, 52 or 73, wherein the fragment or the variant confers a dominant-negative phenotype in the regenerable plant cell;
b. regenerating a transgenic plant from the regenerable plant cell after step (a), wherein the transgenic plant comprises in its genome the recombinant DNA construct; and
c. selecting a transgenic plant of (b), wherein the transgenic plant comprises the recombinant DNA construct and exhibits an alteration of at least one agronomic characteristic selected from the group consisting of: abiotic stress tolerance, early senescence, greenness, biomass, yield, drought tolerance, low nitrogen tolerance, root lodging, harvest index, stalk lodging, plant height, ear height, ear length, salt tolerance, early seedling vigor and seedling emergence under low temperature stress, when compared to a control plant not comprising the recombinant DNA construct.

4. A method of identifying an allele of slm1, the method comprising the steps of:
a. performing a genetic screen on a population of mutant maize plants;
b. identifying one or more mutant maize plants that exhibit a slm1 phenotype; and
c. identifying the slm1 allele from the mutant maize plant with the slm1 phenotype.

5. A method of producing a transgenic plant with alteration of an agronomic characteristic, the method comprising the steps of:
a. crossing a first plant containing a slm1 allele with a second plant containing a recombinant DNA construct comprising an isolated polynucleotide operably linked to a promoter;
b. screening the population of plants from step (a); and
c. selecting a plant comprising the following: (i) the recombinant DNA construct of step (a); (ii) a slm1 phenotype; and (iii) an alteration of at least one agronomic characteristic selected from the group consisting of: abiotic stress tolerance, early senescence, greenness, biomass, yield, drought tolerance, low nitrogen tolerance, root lodging, harvest index, stalk lodging, plant height, ear height, ear length, salt tolerance, early seedling vigor and seedling emergence under low temperature stress, when compared to a control plant comprising the recombinant DNA construct but not comprising the slm1 phenotype.

6. A plant in which expression of the endogenous Slm1 gene is reduced relative to a control plant.

7. A plant or seed produced by the method of claim 5.

8. A method of making the plant of claim 6, the method comprising the steps of
a. introducing a mutation into the endogenous Slm1 gene; and
b. detecting the mutation.

9. The method of claim 8 wherein using the steps (a) and (b) are done using a Targeting Induced Local Lesions IN Genomics (TILLING) method and wherein the mutation is effective in reducing the expression of the endogenous Slm1 gene or its activity, or both.

10. The method of claim 8 wherein the mutation is a site-specific mutation.

11. A method of making the plant of claim 6 wherein the method comprises the steps of:
a. introducing a transposon into a germplasm containing an endogenous Slm1 gene;
b. obtaining progeny of the germplasm of step (a); and
c. identifying a plant of the progeny of step (b) in which the transposon has inserted into the endogenous Slm1 gene and a reduction of expression of Slm1 is observed.

12. The method of claim 11, in which step (a) further comprises introduction of the transposon into a regenerable plant cell of the germplasm by transformation and regeneration of a transgenic plant from the regenerable plant cell, wherein the transgenic plant comprises in its genome the transposon.

13. The method of claim 4 wherein the method further comprises the steps of:
i. introducing into a regenerable plant cell a recombinant DNA construct comprising the slm1 allele identified by the method of claim 4;
ii. regenerating a transgenic plant from the regenerable plant cell after step (i), wherein the transgenic plant comprises in its genome the recombinant DNA construct; and
iii. selecting a transgenic plant of (ii), wherein the transgenic plant comprises the recombinant DNA construct and exhibits a slm1 phenotype, when compared to a control plant not comprising the recombinant DNA construct.

14. The method of claim 3 wherein expression of the polynucleotide of part (a) in a plant line having the les23 mutant genotype is capable of partially or fully restoring the wild-type phenotype.

15. (canceled)

16. (canceled)

17. A plant comprising in its genome a recombinant DNA construct comprising an isolated polynucleotide operably linked, in sense or antisense orientation or both, to a promoter functional in a plant, wherein the polynucleotide comprises:
a. the nucleotide sequence of SEQ ID NO:47, 48, 50 or 51;
b. a nucleotide sequence with at least 90% sequence identity, based on the Clustal W method of alignment, when compared to SEQ ID NO:47, 48, 50 or 51;
c. a nucleotide sequence of at least 100 contiguous nucleotides of SEQ ID NO:47, 48, 50 or 51; or
d. a modified plant miRNA precursor, wherein the precursor has been modified to replace the miRNA encoding region with a sequence designed to produce a miRNA directed to SEQ ID NO:47, 48, 50 or 51; and
wherein the plant exhibits an alteration in at least one agronomic characteristic selected from the group consisting of: abiotic stress tolerance, early senescence, greenness, biomass, yield, drought tolerance, low nitrogen tolerance, root lodging, harvest index, stalk lodging, plant height, ear height, ear length, salt tolerance, early seedling vigor and seedling emergence under low temperature stress, when compared to a control plant not comprising the recombinant DNA construct.

18. The plant of claim 17, wherein said plant is selected from the group consisting of: Arabidopsis, tomato, maize, soybean, sunflower, sorghum, canola, wheat, alfalfa, cotton, rice, barley, millet, sugar cane and switchgrass.

19. Seed of the plant of claim 17, wherein said seed comprises in its genome a recombinant DNA construct comprising an isolated polynucleotide operably linked, in sense or antisense orientation, to a promoter functional in a plant, wherein the polynucleotide comprises:
a. the nucleotide sequence of SEQ ID NO:47, 48, 50 or 51;
b. a nucleotide sequence with at least 90% sequence identity, based on the Clustal W method of alignment, when compared to SEQ ID NO:47, 48, 50 or 51;
c. a nucleotide sequence of at least 100 contiguous nucleotides of SEQ ID NO:47, 48, 50 or 51; or
d. a modified plant miRNA precursor, wherein the precursor has been modified to replace the miRNA encoding region with a sequence designed to produce a miRNA directed to SEQ ID NO:47, 48, 50 or 51; and
wherein a plant produced from the seed exhibits an alteration in at least one agronomic characteristic selected from the group consisting of: abiotic stress tolerance, early senescence, greenness, biomass, yield, drought tolerance, low nitrogen tolerance, root lodging, harvest index, stalk lodging, plant height, ear height, ear length, salt tolerance, early seedling vigor and seedling emergence under low temperature stress, when compared to a control plant not comprising the recombinant DNA construct.

20. A method of identifying a first maize plant or a first maize germplasm that has an alteration of at least one agronomic characteristic, the method comprising detecting in the first maize plant or the first maize germplasm at least one polymorphism of a marker locus that is associated with said agronomic characteristic, wherein the marker locus encodes a polypeptide comprising an amino acid sequence having at least 90% and less than 100% sequence identity to SEQ ID NO:49, wherein expression of said polypeptide in a plant or plant part thereof results in an alteration of at least one agronomic characteristic selected from the group consisting of: abiotic stress tolerance, early senescence, greenness, biomass, yield, drought tolerance, low nitrogen tolerance, root lodging, harvest index, stalk lodging, plant height, ear height, ear length, salt tolerance, early seedling vigor and seedling emergence under low temperature stress, when compared to a control plant, wherein the control plant comprises SEQ ID N0:49.

21. The method of claim 20, wherein said polypeptide comprises the sequence set forth in SEQ ID NO:73.

22. A method of making the plant of claim 6, wherein the method comprises:
a. introducing into a regenerable plant cell a recombinant construct comprising an isolated polynucleotide operably linked to a promoter, wherein the expression of the polynucleotide sequence reduces endogenous Slm1 expression;
b. regenerating a transgenic plant from the regenerable plant cell after step (a), wherein the transgenic plant comprises in its genome the recombinant DNA construct; and
c. selecting the transgenic plant of (b), wherein the transgenic plant comprises the recombinant construct and exhibits a decrease in expression of Slm1, when compared to a control plant not comprising the recombinant DNA construct.

23. A method of making the plant of claim 6, wherein the method comprises:
a. introducing into a regenerable plant cell a recombinant DNA construct comprising an isolated polynucleotide operably linked, sense or antisense orientation, to a promoter functional in a plant, wherein the polynucleotide comprises:
i. the nucleotide sequence of SEQ ID NO:47, 48, 50 or 51;
ii. a nucleotide sequence with at least 90% sequence identity, based on the Clustal W method of alignment, when compared to SEQ ID NO:47, 48, 50 or 51;
iii. a nucleotide sequence of at least 100 contiguous nucleotides of SEQ ID NO:47, 48, 50 or 51;
iv. a nucleotide sequence that can hybridize under stringent conditions with the nucleotide sequence of (i); or
v. a modified plant miRNA precursor, wherein the precursor has been modified to replace the miRNA encoding region with a sequence designed to produce an miRNA directed to SEQ ID NO:47, 48, 50 or 51;
b. regenerating a transgenic plant cell after step (a), wherein the transgenic plant comprises in its genome the recombinant DNA construct; and
c. selecting a transgenic plant of (b), wherein the transgenic plant comprises the recombinant DNA construct and exhibits a decrease in expression of Slm1, when compared to a control plant not comprising the recombinant DNA construct.

24. A method of making the plant of claim 17, wherein the method comprising the steps of:
a. introducing into a regenerable plant cell a recombinant DNA construct comprising an isolated polynucleotide operably linked, in sense or antisense orientation, to a promoter functional in a plant, wherein the polynucleotide comprises:
i. the nucleotide sequence of SEQ ID NO:47, 48, 50 or 51;
ii. a nucleotide sequence with at least 90% sequence identity, based on the Clustal W method of alignment, when compared to SEQ ID NO:47, 48, 50 or 51;
iii. a nucleotide sequence of at least 100 contiguous nucleotides of SEQ ID NO:47, 48, 50 or 51; or
iv. a modified plant miRNA precursor, wherein the precursor has been modified to replace the miRNA encoding region with a sequence designed to produce a miRNA directed to SEQ ID NO:47, 48, 50 or 51;
b. regenerating a transgenic plant from the regenerable plant cell after step (a), wherein the transgenic plant comprises in its genome the recombinant DNA construct; and
c. selecting a transgenic plant of (b), wherein the transgenic plant comprises the recombinant DNA construct and exhibits an alteration in at least one agronomic characteristic selected from the group consisting of: abiotic stress tolerance, early senescence, greenness, biomass, yield, drought tolerance, low nitrogen tolerance, root lodging, harvest index, stalk lodging, plant height, ear height, ear length, salt tolerance, early seedling vigor and seedling emergence under low temperature stress, when compared to a control plant not comprising the recombinant DNA construct.
